SEQUENCE LISTING (18 sequences)

SEQ ID NO: 1 - protein, 19 aa, Artificial Sequence
  Synthetic
MALWMRLLPLLALLALWGP

SEQ ID NO: 2 - protein, 10 aa, Artificial Sequence
  Synthetic
MALWMRLLPL

SEQ ID NO: 3 - protein, 9 aa, Artificial Sequence
  Synthetic
ALLALWGPD

SEQ ID NO: 4 - protein, 34 aa, Artificial Sequence
  Synthetic
AAAFVNQHLCGSHLVEALYLVCGERGFFYTPKTR

SEQ ID NO: 5 - protein, 23 aa, Artificial Sequence
  Synthetic
PAAAFVNQHLCGSHLVEALYLVC

SEQ ID NO: 6 - protein, 13 aa, Artificial Sequence
  Synthetic
PAAAFVNQHLCGS

SEQ ID NO: 7 - protein, 12 aa, Artificial Sequence
  Synthetic
CGSHLVEALYLV

SEQ ID NO: 8 - protein, 8 aa, Artificial Sequence
  Synthetic
VEALYLVC

SEQ ID NO: 9 - protein, 8 aa, Artificial Sequence
  Synthetic
LVCGERGF

SEQ ID NO: 10 - protein, 6 aa, Artificial Sequence
  Synthetic
FFYTPK

SEQ ID NO: 11 - protein, 31 aa, Artificial Sequence
  Synthetic
REAEDLQVGQVELGGGPGAGSLQPLALEGSL

SEQ ID NO: 12 - protein, 12 aa, Artificial Sequence
  Synthetic
REAEDLQVGQVE

SEQ ID NO: 13 - protein, 8 aa, Artificial Sequence
  Synthetic
LGGGPGAG

SEQ ID NO: 14 - protein, 11 aa, Artificial Sequence
  Synthetic
SLQPLALEGSL

SEQ ID NO: 15 - protein, 21 aa, Artificial Sequence
  Synthetic
GIVEQCCTSICSLYQLENYCN

SEQ ID NO: 16 - protein, 14 aa, Artificial Sequence
  Synthetic
GIVEQCCTSICSLY

SEQ ID NO: 17 - protein, 7 aa, Artificial Sequence
  Synthetic
QLENYCN

SEQ ID NO: 18 - protein, 7 aa, Artificial Sequence
  Synthetic
CSLYQLE

(19) United States
(12) Patent Application Publication
(10) Pub. No.: US 2018/0312916 A1
(43) Pub. Date: Nov. 1, 2018

(54) DIGITAL SEQUENCING USING MASS LABELS

(71) Applicants: Mike Joseph Pugia, Ganger, IN (US); Zane Baird, Brigham City, UT (US); Zehui Cao, Carmel, IN (US)

(72) Inventors: Mike Joseph Pugia, Ganger, IN (US); Zane Baird, Brigham City, UT (US); Zehui Cao, Carmel, IN (US)

(21) Appl. No.: 15/953,414

(22) Filed: Apr. 14, 2018

Related U.S. Application Data
(60) Provisional application No. 62/490,094, filed on Apr. 26, 2017.

(51) Int. Cl. C12Q 1/6872

(52) U.S. Cl. CPC C12Q 1/6872

(57) ABSTRACT

The invention provides a means to sequence a gene by mass spectroscopy by release and detection of mass labeled nucleic acids. Mass labels are designed as chain terminators nucleic acid and optimal for ionization by the mass spectrometric method used and there is no loss of sensitivity across genes sequenced and the amplification can be minimized.

This application claims the priority benefit under 35 U.S.C. section 119 of U.S. Provisional Patent Application No. 62/490,094 entitled “Digital Sequencing Using Mass Labels” filed on Apr. 26, 2017; and which is in its entirety herein incorporated by reference.

### BACKGROUND

Historically, mass spectroscopy has been applied to nucleic acid sequencing due to the advantage of being able to unambiguously identify frameshift mutations and heterozygous mutations after re-sequencing. However, using mass spectrometers to analyze the DNA products from Sanger sequencing or enzymatic digestion reactions, the read lengths attainable are currently insufficient for large-scale de novo sequencing.

Several mass spectroscopy methods have been developed to directly sequence DNA by mass spectroscopy using first capture of single strand DNA onto oligonucleotide probe and then amplifying into amplicons. These amplicons can be measured by a mass spectrometer (MS), either by MALDI or ESI, typically MALDI as a complex. The nucleic acid is fragmented inside the mass-spectrometer so the mass of fragment can be related to the theoretical masses of the individual nucleic acids (G, A, C, T, or U). The strength of a nucleotide linkage correlates inversely to its gas phase basicity (G>A, C>T). DNA can also be converted into RNA which is more prone to cleavage in acidic matrices (e.g., DHB) and has higher gas phase stability but still promotes fragmentation into smaller nucleotide structure for analysis. However, the ability of the MS to correctly identify the nucleic acids requires a high mass accuracy, or the ability to read very small changes in mass, typically requiring the more expensive and complex MS analyzers.

The direct sequencing mass spectroscopy method has been shown effective for some sequences and has detected a point mutation in fragment around 10,000 daltons or oligonucleotides of between 25 and 30 nucleotides base pairs (bp) (Braun, A. et. al. Clinical Chem. 43 (1997) 1151). However, as this method relies on the natural nucleotide sequence there are many limitation to sensitivity. Oligonucleotides have a strong tendency to form salt adducts and salts suppresses ionization, reduce signal intensity, reduce mass resolution and increases spectra complexity (Gilar, M. et. al. J. Chromatogr. A 921 (2001) 3). Many have tried to change the ionization solution to get around these problems. For example, desalting by ethanol precipitation (Stults, J. T. et. al. RCMS 5 (1991) 359), organic solvents, organic additives (triethylamine, piperidine, imidazole) and pH 7.0 (Greig, M. et al. RCMS 9 (1995) 97, Smith, R. D. et al. JASMS 1996, 7, 697-706). Others have changed the ESI detection methods as ion are usually multiply charged, making large ions more amenable to quadrupole, ion trap, and FTMS and improving structural accessibility by MS' (n>2). However, all methods are still susceptible to the natural nucleotide sequence and do not work for all sequences at the same sensitivity.

Several new mass spectroscopy methods have been developed to indirectly sequence DNA or RNA. These methods rely on the capture of single strand DNA or RNA onto oligonucleotide probes after amplification. Probe masses can be altered to improve detection. The masses of these probes or their complexes are measured after PCR amplification. The mass spectrometer looks for mass difference in native and mutant form as probes can be designed to only bind to mutations (U.S. Pat. No. 6,949,633, U.S. Pat. No. 7,011,928). The technology works by a process for detecting a target nucleic acid sequence present in a biological sample, comprising: (a) obtaining a nucleic acid molecule from a biological sample; (b) immobilizing the nucleic acid molecule onto a solid support to produce an immobilized nucleic acid molecule; (c) hybridizing detector oligonucleotide with the immobilized nucleic acid molecule and removing unhybridized detector oligonucleotide; (d) ionizing and volatizing the product of (c); and (e) detecting the detector oligonucleotide by mass spectrometry, where detection of the detector oligonucleotide indicates the presence of the target nucleic acid sequence in the sample.

These indirect sequencing methods has been shown to effect 10 and 30 nucleotides base pairs (bp). Here MALDI is less effective for measured duplexes but with use of 6-aza-2-thiothymine (ATT), duplexes of 12-70 bp have been detected. While duplexes as small as 8-bp arecobservable by ESI MS/MS, they vary greatly (Ganem, B. et. al. Tetra. Lett. 34 (1993) 1445 & Bayer, E. et. al. Anal. Chem. 66 (1994) 3858). The charge state of the nucleic acids needs to be reduced with acids like acetic acid, formic acid or TFA to simplify spectra (Smith, R. D. et al. JASMS 1996, 7, 697-706). Measurements of DNA duplexes with small molecules like distamycin A are possible but this does not help provide the sequence (Gale, D. C., et. al. J. Am. Chem. Soc. 116 (1994) 6027).

Additional methods based on matrix-assisted laser desorption/ionization time of flight (MALDI-TOF) mass spectrometry have been developed such as the MassEXTEND method using single allele base extension reaction (SABER), and the allele specific base extension reaction (ASBER) (Gao Top Curr Chem. 2013; 331:55-77, Sharma Int J Mass Spectrom. 2011 July; 304(2-3): 172-183). However, all methods are still susceptible to the nucleotide sequence of the probe or duplex and do not work for all sequences at the same sensitivity.

Sanger sequencing is classical method for gene sequencing and uses a chain-termination method comprised of a single-stranded DNA template, a DNA primer, a DNA polymerase, normal deoxynucleoside triphosphates (dNTPs), and modified di-deoxynucleosidetriphosphates (ddNTPs), the latter of which terminate DNA strand elongation. These ddNTPs or chain-terminating nucleotides lack a 3′-OH group required for the formation of a phosphodiester bond between two nucleotides, causing DNA polymerase to cease extension of DNA when a modified ddNTP is incorporated. The ddNTPs is radioactively or fluorescently labeled for detection in automated sequencing machines.

In practice, a DNA sample is divided into four separate sequencing reactions, containing all four of the standard deoxynucleotides (dATP, dGTP, dCTP and dTTP) and the DNA polymerase. To each reaction, there is added only one of the four dideoxynucleotides (ddATP, ddGTP, ddCTP, or ddTTP), while the other added nucleotides are ordinary ones. The dideoxynucleotide is added to be approximately 100-fold lower in concentration than the corresponding dinucleotide (e.g. 0.005 mM ddATP: 0.5 mM dATP) allowing for enough fragments to be produced while still transcribing the complete sequence. Putting it in a more sensible order, four separate reactions are needed in this process to test all four ddNTPs. Following rounds of template DNA extension from the bound primer, the resulting DNA fragments are heat denatured and separated by size using gel electrophoresis. In the original publication of 1977, the formation of base-paired loops of ssDNA was a cause of serious difficulty in resolving bands at some locations. This method was originally performed using a denaturing polyacrylamide-urea gel with each of the four reactions run in one of four individual lanes (lanes A, T, G, C). The DNA bands may then be visualized by autoradiography or UV light and the DNA sequence can be directly read off the X-ray film or gel image. However, to date no convenient method for Sanger sequencing by mass spectroscopy exist.

Owing to its greater expediency and speed, dye-terminator sequencing is now the mainstay in automated Sanger sequencing. One of the limitations includes dye effects due to differences in the incorporation of the dye-labelled chain terminators into the DNA fragment, resulting in unequal peak heights and shapes in the electronic DNA sequence trace chromatogram after capillary electrophoresis. This dye effect problem has been addressed with the use of modified DNA polymerase enzyme systems and dyes that minimize incorporation variability, as well as methods for eliminating “dye blobs”. The dye-terminator sequencing method, along with automated high-throughput DNA sequence analyzers using “sequencing by synthesis”, are now being used for the vast majority of sequencing projects, however requires too many reads, so called deep sequencing for low purity material.

While sequencing can be done by many molecular approaches including mass spectroscopy (NGS, MS, PCR, and others) for many different types of nucleic acids (RNA or DNA), these methods often generate much too data for simple clinical analysis (e.g 25 million reads at 300 bp read lengths) and have a lot of method steps and complexity needed to handle rare nucleic acid to be sequenced (for example 100,000 reads for a nucleic acid of 0.01% rarity). While mass spectroscopy can detect small reads for a nucleic acid of 0.01% rarity without excessive method steps, the sensitivity varies with the probe, duplex, native sequence or amplicon produced and therefore is prone to false results. A mass spectroscopy method to detect smaller reads for a nucleic acid of 0.01% rarity or less which is not prone to false results would simplify molecular analysis and is a long felt need in the technology.

### SUMMARY OF THE INVENTION

The invention is a means to sequence a gene by mass spectroscopy by release and detection of mass labeled nucleic acids. Mass labels are designed chain terminators nucleic acids and optimal for ionization by the mass spectrometric method used and there is no loss of sensitivity across genes sequenced and the amplification can be minimized.

The key features of this invention are shown in the following steps: (1) isolation of the nucleic acid; (2) amplification of the nucleic acid and chain termination with a releasable mass label terminator such as 2′,3′ dideoxynucleotides (ddNTPs); (3) reading the number of base pairs in products by mass and (4) release of mass label-terminator to identify the terminal nucleotide in the sequence.

This invention works with a nucleic acid that can be identified and measured by release and detection of mass label nucleic acids in several uses. In some examples the nucleic acid is: (1) DNA or RNA isolated by capture and purification; (2) pre-amplification of captured DNA or RNA; (3) DNA or RNA captured on particles or contained inside droplets, and (4) DNA or RNA that is inside cells or released from cells The invention uses mass analysis of mass label released from nucleic acids and mass labels attached to nucleic acids from a liquid holding area for collection and mass spectroscopic analysis. The measure of nucleic acid by mass label can serve as a bar code to identify the presence of unique analytes or as a signal to quantitate the amount of analyte.

### BRIEF DESCRIPTION OF THE DRAWINGS

The drawings provided herein are not to scale and are provided for the purpose of facilitating the understanding of certain examples in accordance with the principles described herein and are provided by way of illustration and not limitation on the scope of the appended claims.

FIG. 1 is a schematic depicting an example of a method in accordance with the principles described herein and shows the process for purification and amplification of DNA and RNA product for digital sequencing (Steps 1). Samples can be DNA or RNA in which is purified and or isolated from cells or from cell free samples. Cellular DNA or RNA undergoes a high-fidelity amplification, whether polymerase replication of DNA to cDNA or reverse transcriptase of RNA to cDNA followed by a targeted capture by nucleic acid affinity particles. Cell free materials undergoes targeted capture by nucleic acid affinity particles prior to the RNA undergoing a target capture on particles followed by a fidelity high-fidelity amplification. This allows enough copies of purified target genes in the form of cDNA which can be analyzed together or separately.

FIG. 2 is another schematic depicting an example of a method in accordance with the principles described herein and shows the process the process for PCR Amplification with MS label terminator and Sanger sequencing and digital MS label read out (Steps 2 and 3). The cDNA undergoes PCR to further amplify the copy number with primer elongation and chain termination. A portion of the amplified product is measured by mass spectroscopy to determine the elongation fragments sizes and a second portion used to release the mass label and determine the terminal nucleotide for each fragment. The combined result allows the sequence to be determined.

FIG. 3 is a further schematic depicting an example of a method in accordance with the principles described herein and shows the examples of chain terminator ddNTP with releaseable mass labels which are releaseable by breaking a bond and can be used to determine base pairs. The mass labels shown uses an acetal bond to releases the mass label at acidic pH. FIG. 3 shows these connections to four base pairs. Mass label can be released and detected in the mass spectrometer. In FIG. 3, R₁, R₂, R₃ and R₄ are alkyl groups having 1-20 carbons.

### DETAILED DESCRIPTION OF THE INVENTION

Methods, apparatus and kits in accordance with the invention described herein have application in any situation where detection or isolation of rare molecules and cells is needed. Examples of such applications include, by way of illustration and not limitation, diagnostics, biological reactions, chemical reactions, high through-put screening, cloning, clone generation, artifical cells, regenerative cells, compound libraries, cell library screening, cell culturing, protein engineering and other applications.

Some examples in accordance with the principles described herein are directed to methods of molecular analysis including compositions and methodologies for sequencing genes using mass spectroscopy techniques. Some examples allow genetic assays for clinical diagnostics and biological studies. Other examples in accordance with the invention described herein are directed to genetic assays for isolation, characterization and detection of cells, particles, macromolecules, genes, proteins, biochemicals, organic molecules or other compounds. Other examples use droplet sorting for detection and genetic analysis of rare cells and cell free molecules. Other examples in accordance with the invention described herein are directed to methods of selective detection of genes, proteins, cells and biomarkers.

Other examples in accordance with the invention described herein are directed to nucleic acid sequencing methods that require binding and separation of cells and cellular biological content whereby cells are isolated on a porous matrix and bound materials retained for analysis. In some cases, the cells are artifical cells, modified cells, natural cells, of any and all types. In other cases the nucleic acids are free of cells.

Some examples in accordance with the invention described herein are directed to methods of binding and separation of nucleic acid, proteins or other biological molecules on to where particles are isolated on a porous matrix or by magnetic particle and bound materials retained for analysis.

Some examples in accordance with the principles described herein are directed to methods of detecting one or more different populations of nucleic acid rare molecules in a sample suspected of containing the one or more different populations of rare molecules and non-rare molecules. These nucleic acids can be used as ligand binding measures of cells, enzymes, proteases, receptors, proteins, nucleic acid, peptidase, proteins, inhibitors and the like by acting on formation or binding of said molecules. These molecules can be formed as metabolites, natural or man-made origin, such as biological, therapeutics, or others.

Examples in accordance with the invention described herein are directed to methods and kits for nucleic acid analysis. Other examples in accordance with the principles described herein are directed to apparatus for analysis.

Common terminology used to describe this invention are “droplet”, “compounds” “in excess”, “rapid”, “emulsion”, “size exclusion filtration”, “compound library”, and are defined further below.

A “droplet” is a micro-bubble defined as a compartment to hold nanoliter (nL) volumes of biological fluidics and compounds. The droplet can contain compounds and be considered “full’. The droplet can lack compounds and be considered “empty”. The “compounds” can be cells, particles, macromolecules, genes, proteins, biochemicals, organic molecules, or others. The droplet size can be varied to reduce the space allowed for a compound, for example the droplet can be nm to μm in diameter. An “excess” of empty droplets to full droplets means a ratio of no greater than 10 full droplets:100 empty droplets such that the ratio of empty to full droplet allows of dilution of sample interference. “Rapid” droplet generation and sorting means at least >10²/sec.

An “emulsion” is created when the droplet separates in two immiscible liquids, namely a generally “aqueous phase” held inside the droplet and a generally “oil phase” outside the droplet. Emulsifiers, surfactants, polar, apolar solvents, solutes and the droplets are considered components of an “emulsion”. The stabilization or destabilization of an “emulsion” can lead to continuation of the “emulsion” or separation of aqueous and oil into separate phases without “droplets”.

“Size exclusion filtration” is the use of a porous matrix to separate droplets and the contents from the rest of the emulsion. The contents of the droplets are retained on the porous matrix and are called “retained contents”. “Retained contents” can be cells or particles and associated molecules. Pore diameters of the porous matrix are kept small enough to retain larger sized droplets and their contents. “Size exclusion filtration” allows washing away unbound material or material not in full droplets or associated with retained contents.

A “library of compounds” is a set of “elements” of a common type including organic molecules, biochemical, genes, particulates, cells, or macromolecules. A “library of compounds” contain any number of unique group members. Generally the library is a group of compounds of similar size and nature and contains some molecule differences between group members. A library of compounds can be a group “variations of peptides and proteins” or variations of nucleic acids such as sequence differences. The “library of compounds” can be captured onto “capture particles”, macromolecules or cells. The “library of compounds” can be captured through an “affinity agent”. Encapsulation of a compound library in a droplet is typically at least 10² different group members.

The term “variations of nucleic acids” is a part, piece, fragment or modification of a nucleic acid of biological or non-biological origin. Binding and association reactions also lead to additional differences in “variations of nucleic acids” as well as a variable domain sequences in gene products.

The term “labeled particle” refers to a particle bound to a mass label agent. This particle can additionally be bound to affinity agents or affinity tags.

The term “capture particle” refers to a particle attached to an affinity agent.

The term “affinity agent” refers to a molecule capable of selectively binding to a specific molecule. The affinity agent can directly bind the rare molecule of interest, the mass label or an affinity tag. The affinity agent can be attached to a capture particle or label particles or can bind a particle through the affinity for the mass label, rare molecule or affinity tag on label particle. The “affinity agent” can be a binding ligand, antigen or substrate to a specific rare molecule.

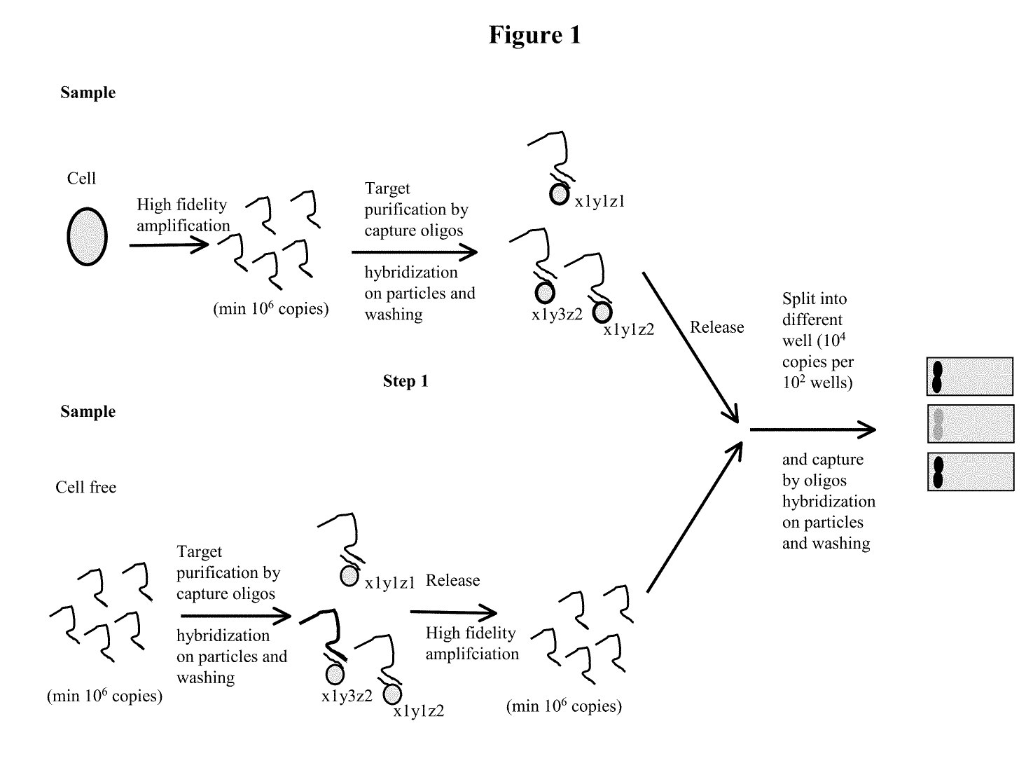

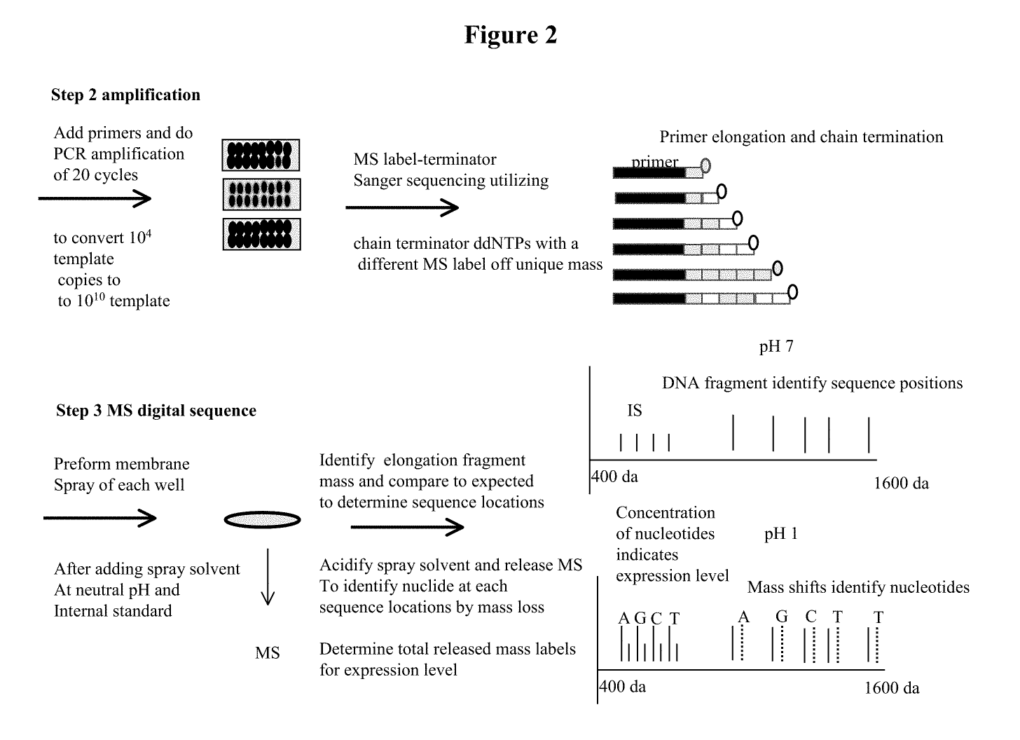

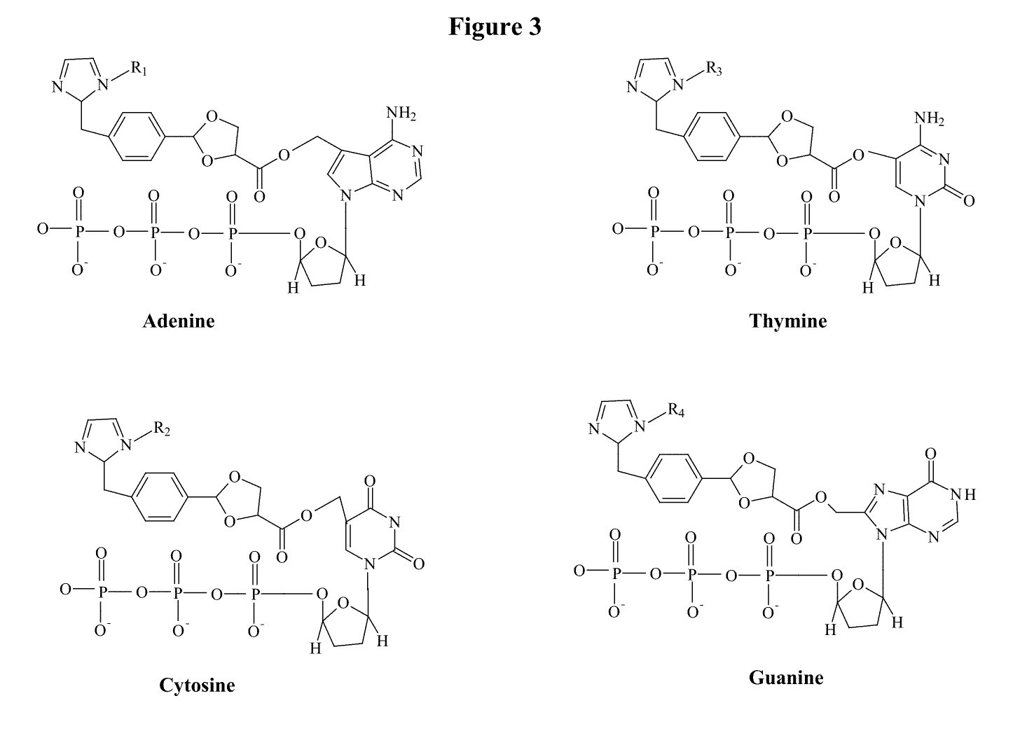

An example of a method for sequencing a gene by mass spectroscopy in accordance with the principles described herein is depicted in FIGS. 1, 2 and 3 and is an example. The principles of a method for sequencing a gene by mass spectroscopy utilize release and detection of mass label from the nucleic acid. Mass labels are designed chain terminators nucleic acid and optimal for ionization by the mass spectrometric used, there is no loss of sensitivity across genes sequenced and the amplification can be minimized.

An example of a method for sequencing follows these steps: (1) isolation of nucleic acid; (2) amplification of nucleic acid and chain termination with a releasable mass label terminator 2′,3′ dideoxynucleotides (ddNTPs) by; (3) reading the number of base pairs in products by mass and (4) release of mass label-terminator for to identify the terminal nucleotide in the sequence.

In some example the nucleic acid that can be identified and measured is of short read lengths, <300 base pairs, or <50 base pairs or only as few as 5 to 50 base pairs such that single point mutations can be identified. In all examples, these nucleic acid contain mass labels. These nucleic acids can be produced by synthesis such as amplification so that mass label-termination occurs.

In some examples, the release of mass label from the terminated chain is used to identify the terminal nucleotide in the sequence. In other examples a digital MS sequencing is achieved by presence or absence reading the base pairs by mass of the mass label. In other examples, the release of mass label is used to identify the terminal nucleotide in the sequence and mass of nucleic acid is used to identify the chain length. In some cases, the presence of only four released mass labels are need to detect nucleic acids of interest. The mass of the sequence is used to identify the chain length of nucleic acid such that chain length of nucleic acid can be read on most spectrometers which have enough resolution to be able to determine the number of base pairs in a nucleic acid and mass labels released.

In other examples, a nucleic acid that can be identified and measured by release and detection of mass label nucleic acids after amplification. In some examples the nucleic acid is: (1) DNA or RNA isolated by capture and purification; (2) pre-amplification of captured DNA or RNA; (3) DNA or RNA captured on particles or contained inside droplets; (4) DNA or RNA captured on particles or contained inside droplets are isolated by size exclusion filtration, (5) DNA or RNA captured on particles and (6) DNA or RNA that is inside cells or released from cells. The invention uses mass analysis of mass label released from nucleic acids and mass labels attached to nucleic acids from a liquid holding area for collection and mass spectroscopic analysis. The measure of nucleic acid by mass label can serve as a bar code to identify the presence of unique analyte or as a signal to quantitate the amount of analyte.

### Examples of Variations of Droplets

A droplet is a micro-bubble defined as a compartment to hold nanoliter (nL)) to microliter (μL) volume of biological fluidics and compounds. The compounds can be organic molecules, biochemical, particles, cells, or other macromolecules. The biological fluidics are aqueous or polar solutions that can contain solutes, polymers, surfactants, emulsifiers, macromolecules, other solvents, and particles in addition to the compounds. The droplet can contain compounds and be considered full. The droplet can lack compounds and be considered empty. The droplet size can be varied to reduce the space allowed for a compound. The droplet size can be varied to reduce the space allowed for a compound, for example the droplet can be varied from 1 to 400 um diameter that hold nL to μL volumes.

The number of empty droplets compared to the number of full droplets can be large (>97%) with small with only (<3%) of droplets created full. In some examples the ratio of full to empty droplets is about 1 to 100, or about 1 to 1000, or about 1 to 10000.

The droplets are made when an emulsion is created by causing the separation of two immiscible liquids, an “aqueous phase” held inside the droplet and a generally “oil phase” outside the droplet. Aqueous phases can include hydrophilic chemical and biochemicals, water, polar protic solvents, polar aprotic solvent and mixtures thereof. The oil phase can include organic solvents, oils such as vegetable, synthetics, animal products, lipids and other lipophilic chemicals and biochemical. The emulsion can be oil-in-water, water in oil, water in oil in water, and oil in water in oil Emulsifiers, emulgents, surfactants can be considered components of the emulsion to change the surface energy of the droplet or the hydrophilic/hydrophobic (lipophilic) balance and include anionic, cationic, nonionic and amphoteric surfactants, as well as naturally occurring materials. Emulsion instability can be caused by sedimentation, aggregation, coalescence and phase inversion. The emulsion stability can be impacted by oil polarity, temperature, nature of solids in the droplet, droplet size and pH. These properties can be used to stabilize or destabilize the droplets and their contents.

The droplets can be made from a feed stock of compound libraries of cells such as rare cells or cell clusters, libraries of particles such as rare molecules on capture particles and labeled particles or libraries of molecules such as genes, proteins, organics and biologics that are isolated as elements into liquid droplets (1 μm to 500 μm diameter). The diameter of the liquid droplets can be adjusted for size of compound libraries, for example the particle size, cell size cluster size, cDNA size and the likes. Each additionally can contain affinity agents and can include labeled nanoparticles either bound to the rare molecules and cells. Additionally, copies of specific cDNA can be reacted onto a specific affinity agent and labeled particle and optionally a capture particle and be contained in the droplet. These labeled particles can serve as indentification markers for genes.

### Examples of Nucleic Acid Amplification or Synthesis Reactions

Droplets can serve as compartments for reactions to produce nucleic acids and nucleic acids with mass labels. For example amplification of isolated material, growth of cells, growth of cell clusters, enzymatic reactions, protein synthesis, metabolism and other biochemical reactions. This can increase the copy number of proteins or molecules from artificial cells so they can be directed for detection, characterization and identification. Additionally, the reactions can replicate genetic material for additional copies or forms, for example reverse transcriptase (RT) reactions to convert RNA to DNA, polymerase chain reactions (PCR), and polymerase (Pol) amplification to make more genetic copies for analysis and convert DNA to cDNA. This can increase the copy number of genetic copier detection, sequencing and archival storage. For example a PCR amplification cane done by adding template to a microwell and allow making 10⁶ product from each copy by, heat at 95 C for 5 min, at 94 C for 1 min, at 60 C for 1 min, at 72 C for 1 min for 20 cycles. In another example, cell free RNA and DNA can be converted to stable cDNA by RT amplification for cell RNA to cDNA and Pol amplification for cfDNA to cDNA. Other example includes cDNA amplicon library preparation for sequencing.

### Examples of Variations of Nucleic Acids

In accordance with the invention described, a “variations of nucleic acids” can be derived from nucleic acids from biological or non-biological origin. The variations of nucleic acids can be used to measure diseases. The variations of nucleic acids can be as a result of disease or intentional reactions. The variations of nucleic acids can result in changes to or from additions of proteins and peptides of man-made or natural origin and include bioactive and non-bioactive peptide or protein. The variations of nucleic acids can be used to measure or produce natural or synthetic molecules such as those used in medical devices, therapeutic use, for diagnostic use, used for measurement of processes, and those used as food, in agriculture, in production, as pro or pre biotics, in microorganism or cellular production, as chemicals for processes, for growth, measurement or control of cells, used for food safety and environmental assessment, used in veterinary products, and used in cosmetics. The nucleic acids can be used to measure enzymes and peptidase of interest based on formation of variations of peptides and proteins. The variations of nucleic acids can be used to measure or produce natural or synthetic inhibitors of enzymes and peptidase inhibitors of interest based on lack of formation of fragments.

The variations of nucleic acids can be used to measure or produce natural or synthetic inhibitors as the result of translation, or posttranslational modification by enzymatic or non-enzymatic modifications or to induce change in cell type, growth or cellular products such as modification of variations of peptides and proteins. Post-translational modification refers to the covalent modification of proteins during or after protein biosynthesis. Post-translational modification can be through enzymatic or non-enzymatic chemical reaction. Phosphorylation is a very common mechanism for regulating the activity of enzymes and is the most common post-translational modification. Enzymes can be oxidoreductases, hydrolases, lyases, isomerases, ligases or transferases as known commonly in enzyme taxomony databases, such as http://enzyme.expasy.org/ or http://www.enzyme-database.org/ which have more than 6000 entries.

Common modification of variations of peptides and proteins include the addition of hydrophobic groups for membrane localization, addition of cofactors for enhanced enzymatic activity, diphthalamide formation, hypusine formation, ethanolamine phosphoglycerol attachment, diphthamide formation, acylation, alkylation amide bond formation such as amino acid addition or amidation, butyrylation gamma-carboxylation dependent on Vitamin K[15], glycosylation, the addition of a glycosyl group to either arginine, asparagine, cysteine, hydroxylysine, serine, threonine, tyrosine, or tryptophan resulting in a glycoprotein, malonylationhydroxylation, iodination, nucleotide addition such as ADP-ribosylation, phosphate ester (O-linked) or phosphoramidate (N-linked) formation such as phosphorylation or adenylylation, propionylation, pyroglutamate formation, S-glutathionylation, S-nitrosylation S-sulfenylation (aka S-sulphenylation, succinylation or sulfation. Nonenzymatic modification include the attachment of sugars, carbamylation, carbonylation or intentional recombinate or synthetic conjugation such as biotinylation or addition of affinity tags, like His oxidation, formation of disulfide bonds between Cys residues or pegylation.

### Examples of Affinity Agent

An affinity agent is a molecule capable of binding selectively to a rare molecule or mass labels. Selective binding involves the specific recognition of one of two different molecules for the other compared to substantially less recognition of other molecules. The terms “binding” or “bound” refers to the manner in which two moieties are associated with one another.

An affinity agent can be an immunoglobulin, protein, peptide, metal, carbohydrate, metal chelator, nucleic acid or other molecule capable of binding selectively to a particular rare molecule or a mass labels type. Selective binding involves the specific recognition of one of two different molecules for the other compared to substantially less recognition of other molecules.

Examples of nucleic acid affinity agents include but are not limited to natural or made-made oligomeric nucleic acids. The oligomeric nucleic acid may be any polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, silencing (siRNA), xeno-nucleic acids (XNA), recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer.

The sequence of nucleotides may be interrupted by non-nucleotide components. A polynucleotide may be further modified, such as by conjugation with a labeling component. The terms “isolated nucleic acid” and “isolated polynucleotide” are used interchangeably; a nucleic acid or polynucleotide is considered “isolated” if it: (1) is not associated with all or a portion of a polynucleotide in which the “isolated polynucleotide” is found in nature, (2) is linked to a polynucleotide to which it is not linked in nature, or (3) does not occur in nature as part of a larger sequence.

The affinity agents which are immunoglobulins which bind nucleic acids may include a complete antibodies or fragments thereof, which immunoglobulins include the various classes and isotypes, such as IgA, IgD, IgE, IgG1, IgG2a, IgG2b and IgG3, IgM, etc. Fragments thereof may include Fab, Fv and F(ab′)2, and Fab′, for example. In addition, aggregates, polymers, and conjugates of immunoglobulins or their fragments can be used where appropriate so long as binding affinity for a particular molecule is maintained. Antibodies can be monoclonal or polyclonal. Such antibodies can be prepared by techniques that are well known in the art such as immunization of a host and collection of sera (polyclonal) or by preparing continuous hybrid cell lines and collecting the secreted protein (monoclonal) or by cloning and expressing nucleotide sequences or mutagenized versions thereof coding at least for the amino acid sequences required for specific binding of natural antibodies.

Polyclonal antibodies and monoclonal antibodies may be prepared by techniques that are well known in the art. For example, in one approach monoclonal antibodies are obtained by somatic cell hybridization techniques. Monoclonal antibodies may be produced according to the standard techniques of Köhler and Milstein, Nature 265:495-497, 1975. Reviews of monoclonal antibody techniques are found in Lymphocyte Hybridomas, ed. Melchers, et al. Springer-Verlag (New York 1978), Nature 266: 495 (1977), Science 208: 692 (1980), and Methods of Enzymology 73 (Part B): 3-46 (1981). In general, monoclonal antibodies can be purified by known techniques such as, but not limited to, chromatography, e.g., DEAE chromatography, ABx chromatography, and HPLC chromatography; and filtration, for example.

An affinity agent can additionally be a “cell affinity agent” capable of binding selectively to a rare molecule which is used for typing a rare cell or measuring a biological intracellular process of a cell. These rare cell markers can be immunoglobulins that specifically recognizes and binds to an antigen associated with a particular cell type and whereby antigen are components of the cell. The cell affinity agent is capable of being absorbed into or onto the cell. The term “cell affinity agent” refers to a rare cell typing markers capable of binding selectively to rare cell. Selective cell binding typically involves “binding between molecules that is relatively dependent of specific structures of binding pair. Selective binding does not rely on non-specific recognition.

### Examples Label and Capture Particles

Affinity agents can be attached to mass labels and/or particles for purpose of detection or isolation of rare molecules. This attachment can occur through “labeled particles” which are in turn attached mass labels. Affinity agents can also be attached to “capture particles” which allow separation of bound and unbound mass labels or rare molecule. This attachment to capture and label can be prepared by directly attaching the affinity agent in a “linking group”. The terms “attached” or “attachment” refers to the manner in which two moieties are connected and accomplished by a direct bond between the two moieties or a linking group between the two moieties. This allows the method to be multiplexed for more than one result at a time. Alternatively, affinity agent can be attached to mass labels and/or particles using additional “binding partners”. The phrase “binding partner” refers to a molecule that is a member of a specific binding pair of affinity agent and “affinity tags” that bind each other and not the mass labels or rare molecules. In some cases, the affinity agent may be members of an immunological pair such as antigen to antibody or hapten to antibody, biotin to avidin, IgG to protein A, secondary antibody to primary antibody, antibodies to fluorescent labels and other examples binding pairs.

The “labeled particle” is a particulate material which can be attached to the affinity agent through a direct linker arm or a binding pair. Also the “labeled particle” is capable of forming an X-Y cleavable linkage between labeled particle and mass label. The size of the label particle is large enough to accommodate one or more mass labels and affinity agent. The ratio of affinity agents or mass label to a single label particle may be 10⁷ to 1, 10⁶ to 1, or 10⁵ to 1, or 10⁴ to 1, or 10³ to 1, or 10² to 1, or 10 to 1, for example. The number of affinity agents and mass labels associated with the label particle is dependent on one or more of the nature and size of the affinity agent, the nature and size of the labeled particle, the nature of the linker arm, the number and type of functional groups on the label particle, and the number and type of functional groups on the mass label, for example.

The composition of the label or capture particle entity may be organic or inorganic, magnetic or non-magnetic as a nanoparticle or a micro particle. Organic polymers include, by way of illustration and not limitation, nitrocellulose, cellulose acetate, poly(vinyl chloride), polyacrylamide, polyacrylate, polyethylene, polypropylene, poly(4-methylbutene), polystyrene, poly(methyl methacrylate), poly(hydroxyethyl methacrylate), poly(styrene/divinylbenzene), poly(styrene/acrylate), poly(ethylene terephthalate), dendrimer, melamine resin, nylon, poly(vinyl butyrate), for example, either used by themselves or in conjunction with other materials and including latex, microparticle and nanoparticle forms thereof. The particles may also comprise carbon (e.g., carbon nanotubes), metal (e.g., gold, silver, and iron, including metal oxides thereof), colloids, dendrimers, dendrons, and liposomes, for example. In some examples, the labeled particle may be a silica nanoparticle. In other examples, labeled particles can be magnetic that have free carboxylic acid, amine or tosyl groups. In other some examples, labeled particles can be mesoporous and include mass labels inside the labeled particles.

The diameter of the labeled or capture particle is dependent on one or more of the nature of the rare molecule, the nature of the sample, the permeability of the cell, the size of the cell, the size of the nucleic acid, the size of the affinity agent, the magnetic forces applied for separation, the nature and the pore size of a filtration matrix, the adhesion of the particle to matrix, the surface of the particle, the surface of the matrix, the liquid ionic strength, liquid surface tension and components in the liquid, and the number, size, shape and molecular structure of associated label particles, for example.

The term “permeability” means the ability of a particles and molecule to enter a cell through the cell wall. In the case of detection of a rare molecule inside the cell, the diameter of the label particles must be small enough to allow the affinity agents to enter the cell. The label particle maybe coated with materials to increase “permeability” like collagenase, peptides, proteins, lipid, surfactants, and other chemicals known to increase particle inclusion into the cell.

When a porous matrix is employed in a filtration separation step, the diameter of the label particles must be small enough to be pass through the pores of a porous matrix if it did bind the rare molecule, and the diameter of the label particles must be large enough to not pass through the pores of a porous matrix to retain the bound rare molecule on the matrix. In some examples in accordance with the principles described herein, the average diameter of the label particles should be at least about 0.01 microns (10 nm) and not more than about 10 microns In some examples, the particles have an average diameter from about 0.02 microns to about 0.06 microns, or about 0.03 microns to about 0.1 microns, or about 0.06 microns to about 0.2 microns, or about 0.2 microns to about 1 micron, or about 1 micron to about 3 microns, or about 3 micron to about 10 microns. In some examples, the adhesion of the particles to the surface is so strong that the particle diameter can be smaller than the pore size of the matrix.

The affinity agent can be prepared by directly attaching the affinity agent to carrier or capture particles by linking groups. The linking group between the label particle and the affinity agent, may be aliphatic or aromatic bond. The linking groups may comprise a cleavable or non-cleavable linking moiety. Cleavage of the cleavable moiety can be achieved by electrochemical reduction used for the mass label but also may be achieved by chemical or physical methods, involving further oxidation, reduction, solvolysis, e.g., hydrolysis, photolysis, thermolysis, electrolysis, sonication, and chemical substitution, for example. Photocleavable bonds that are cleavable with light having an appropriate wavelength such as, e.g., UV light at 300 nm or greater; for example. The nature of the cleavage agent is dependent on the nature of the cleavable moiety. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers, ureas, thioureas, guanidines, azo groups, thioethers, carboxylate and so forth. The linking group may also be a macro-molecule such as polysaccharides, peptides, proteins, nucleotides, and dendrimers.

The linking group between the particle and the affinity agent may be a chain of from 1 to about 60 or more atoms, or from 1 to about 50 atoms, or from 1 to about 40 atoms, or from 1 to 30 atoms, or from about 1 to about 20 atoms, or from about 1 to about 10 atoms, each independently selected from the group normally consisting of carbon, oxygen, sulfur, nitrogen, and phosphorous, usually carbon and oxygen. The number of heteroatoms in the linking group may range from about 0 to about 8, from about 1 to about 6, or about 2 to about 4. The atoms of the linking group may be substituted with atoms other than hydrogen such as, for example, one or more of carbon, oxygen and nitrogen in the form of, e.g., alkyl, aryl, aralkyl, hydroxyl, alkoxy, aryloxy, or aralkoxy groups. As a general rule, the length of a particular linking group can be selected arbitrarily to provide for convenience of synthesis with the proviso that there is minimal interference caused by the linking group with the ability of the linked molecules to perform their function related to the methods disclosed herein.

Obtaining reproducibility in amounts of particle captured after separation and isolation is important for rare molecular analysis. Additionally, the amounts of particle captured that enter a rare cell is important to maximize the amount of specific binding. Knowing the amount of particles remaining after washing are important to minimize the amount of non-selective binding. In order to make these determination, it is helpful if the particles can contain fluorescent, optical or chemiluminescence labels. Therefore, labeled particles, can be measured by fluorescent or chemiluminescence by virtue of the presence of a fluorescent or chemiluminescence molecule. The fluorescent and optical molecule can then be measured by microscopic analysis and compared to expected results for sample containing and lacking analyte. Fluorescent molecule include but are not limited to Dylight™, FITC, rhodamine compounds, phycoerythrin, phycocyanin, allophycocyanin, o phthalaldehyde, fluorescent rare earth chelates, amino-coumarins, umbelliferones, oxazines, Texas red, acridones, perylenes, indacines such as, e.g., 4,4-difluoro-4-bora-3a,4a-diaza-s-indacene and variants thereof, 9,10-bis-phenylethynyl-anthracene, squarine dyes and fluorescamine, for example. A fluorescent microscope or fluorescent spectrometer may then be used to determine the location and amount of the labeled particles. Chemiluminescence labels examples include luminol, acridinium esters and acridinium sulfonamides to name a few. Optical labels examples include color particles, gold particles, enzymatic colorimetric reactions to name a few.

### Examples of Porous Matrix and Filtration

Porous matrices are used in “size exclusion filtration” to allow washing away unbound material or material not in full droplets or associated with retained contents. The contents of the droplets are retained on the porous matrix and are called “retained contents”. “Retained contents” can be cells or particles and molecules associated. Full droplets can also be retained with contents on the porous matrix. Pore diameters of the porous matrix are kept small enough to retain larger sized droplets and their contents. “Size exclusion filtration” allows washing away unbound material or material not in full droplets or associated with retained contents.

Porous matrix can be at bottom of a liquid well to hold the droplets and retained contents on cells and particles. Well diameters must be greater than droplets, cell or particles used to reaction the content in a well while still not obstructing washing and allowing washing away undesired materials. Droplet diameter can vary from 1 to 400 μm. Particle size diameter can vary from 15 nm to 10 μm and serve as capture or detection particles. Particles can be associated with other particle or cells. Detection particle and cells or capture particle isolation can be used for the detection of rare molecule. Porous matrices are used where the detection of particles are sufficiently smaller than the pore size of the matrix such that physically the particles can fall through the pores if not captured. In other examples, the capture particles are sufficiently larger than the pore size of the matrix such that physically the particles cannot fall through the pores. Cells size diameters can vary from 1 μm to 50 μm. Cells can also be in clusters or spheroids of multiple cells of up to an average diameter of 200 μm. The ratio of well diameter is at least 2 times greater than the diameter of droplet, cells, cell clusters or spheroids. This allows individual droplet, cells, cell clusters or spheroids in a well. The ratio of droplet or cells is less than 10 to improve separation of one droplet or cells per well.

In some methods in accordance with the principles described herein, the sample is incubated with an affinity agent comprised of a mass label and labeled particle, for each different population of rare molecules. The affinity agent that comprises a specific binding partner that is specific for and binds to a rare molecule of one of the populations of the rare molecules. The rare molecules can be cell bound or cell free. The affinity agent with mass label and labeled particle are retained on the surface of a membrane after a filtration.

The separation can occur as in some examples when a porous matrix employed in a filtration separation step is such that the pore diameter is smaller than the diameter of the cell with the rare molecule but larger that the unbound labeled particles to allow the affinity agents to achieve the benefits of rare molecule capture in accordance with the principles described herein but small enough to be pass through the pores of a porous matrix or if it did not capture rare molecule. In other methods, the porous matrix employed in a filtration separation step is such that the pore diameter is smaller than the diameter of the affinity agents on labeled particle capable of binding rare molecule but larger that the unbound molecule pass through allowing the affinity agents to achieve the benefits of rare molecule capture. In still other methods, the affinity agents on labeled particle can be additionally bound through “binding partners” or sandwich assays to other capture particles, like magnetic particles, or to a surface, like a membrane. In the later case, the capture particles are retained on the surface of the porous membranes.

In all examples, the concentration of one or more different populations of rare molecules is enhanced over that of the non-rare molecules to form a concentrated sample. In some examples, the sample is subjected to a filtration procedure using a porous matrix that retains the rare molecules while allowing the non-rare molecules to pass through the porous matrix thereby enhancing the concentration of the rare molecules. In the event that one or more rare molecules are non-cellular, i.e., not associated with a cell or other biological particle, the sample is combined with one or more capture particle entities wherein each capture particle entity comprises a binding partner for the non-cellular rare molecule of each of the populations of non-cellular rare molecules to render the non-cellular rare molecules in particulate form, i.e., to form particle-bound non-cellular rare molecules. The combination of the sample and the capture particle entities is held for a period of time and at a temperature to permit the binding of non-cellular rare molecules with corresponding binding partners of the capture particle entities.

Vacuum is applied to the sample on the porous matrix to facilitate passage of non-rare cells and other particles through the matrix. The level of vacuum applied is dependent on one or more of the nature and size of the different populations of rare cells and/or particle reagents, the nature of the porous matrix, and the size of the pores of the porous matrix, for example.

Contact of the sample with the porous matrix is continued for a period of time sufficient to achieve retention of cellular rare molecules and/or particle-bound non-cellular rare molecules on a surface of the porous matrix to obtain a surface of the porous matrix having different populations of rare cells and/or particle-bound rare molecules as discussed above. The period of time is dependent on one or more of the nature and size of the different populations of rare cells and/or particle-bound rare molecules, the nature of the porous matrix, the size of the pores of the porous matrix, the level of vacuum applied to the sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

An amount of each different affinity agent that is employed in the methods in accordance with the principles described herein is dependent on one or more of the nature and potential amount of each different population of rare molecules, the nature of the mass label, the natured of attachment, the nature of the affinity agent, the nature of a cell if present, the nature of a particle if employed, and the amount and nature of a blocking agent if employed, for example. In some examples, the amount of each different modified affinity agent employed is about 0.001 μg/μL to about 100 μg/μL, or about 0.001 μg/μL to about 80 μg/μL, or about 0.001 μg/μL to about 60 μg/μL, or about 0.001 μg/μL to about 40 μg/μL, or about 0.001 μg/μL to about 20 μg/μL, or about 0.001 μg/μL to about 10 μg/μL, or about 0.5 μg/μL to about 100 μg/μL, or about 0.5 μg/μL to about 80 μg/μL, or about 0.5 μg/μL, to about 60 μg/μL, or about 0.5 μg/μL to about 40 μg/μL, or about 0.5 μg/μL to about 20 μg/μL, or about 0.5 μg/μL to about 10 μg/μL, for example.

The porous matrix is a solid, material, which is impermeable to liquid (except through one or more pores of the matrix is in accordance with the invention described herein. The porous matrix is associated with a porous matrix holder and a liquid holding well. The association between porous matrix and holder can be done with an adhesive. The association between porous matrix in the holder and the liquid holding well can be through direct contact or with a flexible gasket surface.

The porous matrix is a solid or semi-solid material and may be comprised of an organic or inorganic, water insoluble material. The porous matrix is non-bibulous, which means that the membrane is incapable of absorbing liquid. In some examples, the amount of liquid absorbed by the porous matrix is less than about 2% (by volume), or less than about 1%, or less than about 0.5%, or less than about 0.1%, or less than about 0.01%, or 0%. The porous matrix is non-fibrous, which means that the membrane is at least 95% free of fibers, or at least 99% free of fibers, or at least 99.5%, or at least 99.9% free of fibers, or 100% free of fibers.

The porous matrix can have any number of shapes such as, for example, track-etched, or planar or flat surface (e.g., strip, disk, film, matrix, and plate). The matrix may be fabricated from a wide variety of materials, which may be naturally occurring or synthetic, polymeric or non-polymeric. The shape of the porous matrix is dependent on one or more of the nature or shape of holder for the membrane, of the microfluidic surface, of the liquid holding well, of cover surface, for example. In some examples the shape of the porous matrix is circular, oval, rectangular, square, track-etched, planar or flat surface (e.g., strip, disk, film, membrane, and plate), for example.

The porous matrix and holder may be fabricated from a wide variety of materials, which may be naturally occurring or synthetic, polymeric or non-polymeric. Examples, by way of illustration and not limitation, of such materials for fabricating a porous matrix include plastics such as, for example, polycarbonate, poly (vinyl chloride), polyacrylamide, polyacrylate, polyethylene, polypropylene, poly-(4 methylbutene), polystyrene, polymethacrylate, poly-(ethylene terephthalate), nylon, poly(vinyl butyrate), poly(chlorotrifluoroethylene), poly(vinyl butyrate), polyimide, polyurethane, and parylene, silanes, silicon, silicon nitride, graphite, ceramic material (such, e.g., as alumina, zirconia, PZT, silicon carbide, aluminum nitride), metallic material (such as, e.g., gold, tantalum, tungsten, platinum, and aluminum); glass (such as, e.g., borosilicate, soda lime glass, and PYREX®); and bioresorbable polymers (such as, e.g., poly-lactic acid, polycaprolactone and polyglycolic acid); for example, either used by themselves or in conjunction with one another and/or with other materials. The material for fabrication of the porous matrix and holder are non-bibulous and does not include fibrous materials such as cellulose (including paper), nitrocellulose, cellulose acetate, rayon, diacetate, lignins, mineral fibers, fibrous proteins, collagens, synthetic fibers (such as nylons, dacron, olefin, acrylic, polyester fibers, for example) or, other fibrous materials (glass fiber, metallic fibers), which are bibulous and/or permeable and, thus, are not in accordance with the principles described herein. The material for fabrication of the porous matrix and holder may be the same or different materials.

The porous matrix for each liquid holding well comprises at least one pore and no more than about 2,000,000 pores per square centimeter (cm²). In some examples, the number of pores of the porous matrix per cm² is 1 to about 2,000,000, or 1 to about 1,000,000, or 1 to about 500,000, or 1 to about 200,000, or 1 to about 100,000, or 1 to about 50,000, or 1 to about 25,000, or 1 to about 10,000, or 1 to about 5,000, or 1 to about 1,000, or 1 to about 500, or 1 to about 200, or 1 to about 100, or 1 to about 50, or 1 to about 20, or 1 to about 10, or 2 to about 500,000, or 2 to about 200,000, or 2 to about 100,000, or 2 to about 50,000, or 2 to about 25,000, or 2 to about 10,000, or 2 to about 5,000, or 2 to about 1,000, or 2 to about 500, or 2 to about 200, or 2 to about 100, or 2 to about 50, or 2 to about 20, or 2 to about 10, or 5 to about 200,000, or 5 to about 100,000, or 5 to about 50,000, or 5 to about 25,000, or 5 to about 10,000, or 5 to about 5,000, or 5 to about 1,000, or 5 to about 500, or 5 to about 200, or 5 to about 100, or 5 to about 50, or 5 to about 20, or 5 to about 10, for example. The density of pores in the porous matrix is about 1% to about 20%, or about 1% to about 10%, or about 1% to about 5%, or about 5% to about 20%, or about 5% to about 10%, for example, of the surface area of the porous matrix. In some examples, the size of the pores of a porous matrix is that which is sufficient to preferentially retain liquid while allowing the passage of liquid droplets formed in accordance with the principles described herein. The size of the pores of the porous matrix is dependent on the nature of the liquid, the size of the cell, the size of the capture particle, the size of mass label, the size of an analyte, the size of labeled particles, the size of non-rare molecules, and the size of non-rare cells, for example. In some examples the average size of the pores of the porous matrices about 0.1 to about 20 microns, or about 0.1 to about 5 microns, or about 0.1 to about 1 micron, or about 1 to about 20 microns, or about 1 to about 5 microns, or about 1 to about 2 microns, or about 5 to about 20 microns, or about 5 to about 10 microns, for example.

Pores within the matrix may be fabricated in accordance with the invention described herein may be fabricated by, for example, by microelectromechanical (MEMS) technology, metal oxide semiconductor (CMOS) technology, micro-manufacturing processes for producing microsieves, laser technology, irradiation, molding, and micromachining, for example, or a combination thereof.

The porous matrix is permanently attached to a holder which can be associated to the bottom of a liquid holding well and to the top of the vacuum manifold where the porous matrix is positioned such that liquid can flow from liquid holding well to vacuum manifold. In some examples, the porous matrix in the holder can be associated to microfluidic surface, top or bottom cover surface. The holder may be constructed of any suitable material that is compatible with the material of the porous matrix. Examples of such materials include, by way of example and not limitation, any of the materials listed above for the porous matrix. The material for the housing and for the porous matrix may be the same or may be different. The holder may also be constructed of non-porous glass or plastic film.

Examples of plastic film materials include polystyrene, polyalkylene, polyolefins, epoxies, Teflon®, PET, chloro-fluoroethylenes, polyvinylidene fluoride, PE-TFE, PE-CTFE, liquid crystal polymers, Mylar®, polyester, polymethylpentene, polyphenylene sulfide, and PVC plastic films. The plastic film can be metallized such as with aluminum. The plastic films can have relative low moisture transmission rate, e.g. 0.001 mg per m²-day. The porous matrix may be permanently fixed attached to a holder by adhesion using thermal bonding, mechanical fastening or through use of permanent adhesives such as drying adhesive like polyvinyl acetate, pressure-sensitive adhesives like acrylate-based polymers, contact adhesives like natural rubber and polychloroprene, hot melt adhesives like ethylene-vinyl acetates, and reactive adhesives like polyester, polyol, acrylic, epoxies, polyimides, silicones rubber-based and modified acrylate and polyurethane compositions, natural adhesive like dextrin, casein and lignin. The plastic film or the adhesive can be electrically conductive materials and the conductive material coatings or materials can be patterned across specific regions of the hold surface.

The porous matrix in the holder is generally part of a filtration module where the porous matrix is part of an assembly for convenient use during filtration. The holder does not contain pores and has a surface which facilitates contact with associated surfaces but is not permanently attached to these surfaces and can be removed. A top gasket maybe applied to the removable holder between the liquid holding wells. A bottom gasket maybe applied to the removable holder between the manifold for vacuum. A gasket is a flexible material that facilities complete contact upon compression. The holder maybe constructed of gasket material. Examples of gasket shapes include a flat, embossed, patterned, or molded sheets, rings, circles, ovals, with cut out areas to allow sample to flow from porous matrix to vacuum maniford. Examples of gasket materials include paper, rubber, silicone, metal, cork, felt, neoprene, nitrile rubber, fiberglass, polytetrafluoroethylene like PTFE or Teflon or a plastic polymer like polychlorotrifluoro-ethylene.

In some examples, vacuum is applied to the concentrated and treated sample on the porous matrix to facilitate passage of non-rare cells through the matrix. The level of vacuum applied is dependent on one or more of the nature and size of the different populations of biological particles, the nature of the porous matrix, and the size of the pores of the porous matrix, for example. In some examples, the level of vacuum applied is about 1 millibar to about 100 millibar, or about 1 millibar to about 80 millibar, or about 1 millibar to about 50 millibar, or about 1 millibar to about 40 millibar, or about 1 millibar to about 30 millibar, or about 1 millibar to about 25 millibar, or about 1 millibar to about 20 millibar, or about 1 millibar to about 15 millibar, or about 1 millibar to about 10 millibar, or about 5 millibar to about 80 millibar, or about 5 millibar to about 50 millibar, or about 5 millibar to about 30 millibar, or about 5 millibar to about 25 millibar, or about 5 millibar to about 20 millibar, or about 5 millibar to about 15 millibar, or about 5 millibar to about 10 millibar, for example. In some examples the vacuum is an oscillating vacuum, which means that the vacuum is applied intermittently at regular of irregular intervals, which may be, for example, about 1 second to about 600 seconds, or about 1 second to about 500 seconds, or about 1 second to about 250 seconds, or about 1 second to about 100 seconds, or about 1 second to about 50 seconds, or about 10 seconds to about 600 seconds, or about 10 seconds to about 500 seconds, or about 10 seconds to about 250 seconds, or about 10 seconds to about 100 seconds, or about 10 seconds to about 50 seconds, or about 100 seconds to about 600 seconds, or about 100 seconds to about 500 seconds, or about 100 seconds to about 250 seconds, for example. In this approach, vacuum is oscillated at about 0 millibar to about 10 millibar, or about 1 millibar to about 10 millibar, or about 1 millibar to about 7.5 millibar, or about 1 millibar to about 5.0 millibar, or about 1 millibar to about 2.5 millibar, for example, during some or all of the application of vacuum to the sample. Oscillating vacuum is achieved using an on-off switch, for example, and may be conducted automatically or manually.

Contact of the treated sample with the porous matrix is continued for a period of time sufficient to achieve retention of the rare cells or the particle-bound rare molecules on a surface of the porous matrix to obtain a surface of the porous matrix having different populations of rare cells or the particle-bound rare molecules as discussed above. The period of time is dependent on one or more of the nature and size of the different populations of rare cells or particle-bound rare molecules, the nature of the porous matrix, the size of the pores of the porous matrix, the level of vacuum applied to the sample on the porous matrix, the volume to be filtered, and the surface area of the porous matrix, for example. In some examples, the period of contact is about 1 minute to about 1 hour, about 5 minutes to about 1 hour, or about 5 minutes to about 45 minutes, or about 5 minutes to about 30 minutes, or about 5 minutes to about 20 minutes, or about 5 minutes to about 10 minutes, or about 10 minutes to about 1 hour, or about 10 minutes to about 45 minutes, or about 10 minutes to about 30 minutes, or about 10 minutes to about 20 minutes, for example.

### Examples of Rare Molecules

The phrase “rare molecules” refers to a molecule that may be detected in a sample where the rare molecules are indicative of a particular population of molecules. The phrase “population of molecules” refers to a group of rare molecules that share a common rare molecules that is specific for the group of rare molecules. The phrase “specific for” means that the common rare molecules distinguishes the group of rare molecules from other molecules.

The methods described herein involve trace analysis, i.e., minute amounts of material on the order of 1 to about 100,000 copies of rare cells or rare molecules. Since this process involves trace analysis at the detection limits of the nucleic acid analyzers, these minute amounts of material can only be detected when detection volumes are extremely low, for example, 10-15 liter, so that the concentrations are within the detection limits. Given associated errors is unlikely and that “all” of the rare molecules undergo amplification, i.e., converting the minute amounts of material to the order of about 10⁵ to about 10¹⁰ copies of every rare molecule. The phrase “substantially all” means that at least about 70 to about 99% measured by the reproducibility of the amount of a rare molecule produced.

The phrase “cell free rare molecules” refers to rare molecules that are not bound to a cell and/or that freely circulate in a sample. Such non-cellular rare molecules include biomolecules useful in medical diagnosis and treatments of diseases. Medical diagnosis of diseases include, but are not limited to, biomarkers for detection of cancer, cardiac damage, cardiovascular disease, neurological disease, hemostasis/hemastasis, fetal maternal assessment, fertility, bone status, hormone levels, vitamins, allergies, autoimmune diseases, hypertension, kidney disease, metabolic disease, diabetes, liver diseases, infectious diseases and other biomolecules useful in medical diagnosis of diseases, for example.

The following are non-limiting examples of samples that rare molecules that can be measured according to the invention. The sample to be analyzed is one that is suspected of containing rare molecules. The samples may be biological samples or non-biological samples. Biological samples may be from a plant, animal, protists or other living organism including animalia, fungi, plantae, chromista, or protozoa or other eukaryote species or bacteria, archaea, or other prokaryote species. Non-biological samples include aqueous solutions, environmental, products, chemical reaction production, waste streams, foods, feed stocks, fertilizers, fuels, and the like. Biological samples include biological fluids such as whole blood, serum, plasma, sputum, lymphatic fluid, semen, vaginal mucus, feces, urine, spinal fluid, saliva, stool, cerebral spinal fluid, tears, mucus, or tissues for example. Biological tissue includes, by way of illustration, hair, skin, sections or excised tissues from organs or other body parts, for example Rare molecules may be from tissues, for example, lung, bronchus, colon, rectum, extra cellular matrix, dermal, vascular, stem, lead, root, seed, flower, pancreas, prostate, breast, liver, bile duct, bladder, ovary, brain, central nervous system, kidney, pelvis, uterine corpus, oral cavity or pharynx or cancers. In many instances, the sample is aqueous such as a urine, whole blood, plasma or serum sample, in other instances the sample must be made into a solution or suspension for testing.

The sample can be one that contains cells such as, for example, non-rare cells and rare cells where rare molecules are detected from the rare cells. The rare molecules from cells may be from any organism, but are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesenchymal cells; circulating epithelial cells; fetal cells; immune cells (B cells, T cells, macrophages, NK cells, monocytes); and stem cells; for example. In other examples of methods in accordance with the principles described herein, the sample to be tested is a blood sample from a organism such as, but not limited to, a plant or animal subject, for example. In some examples of methods in accordance with the principles described herein, the sample to be tested is a sample from a organism such as, but not limited to, a mammal subject, for example. Cells with rare molecules may be from a tissue of mammal, for example, lung, bronchus, colon, rectum, pancreas, prostate, breast, liver, bile duct, bladder, ovary, brain, central nervous system, kidney, pelvis, uterine corpus, oral cavity or pharynx or cancers.

Rare molecule fragments can be used to measure peptidases of interest including those in the MEROPS on-line database for peptidases (also known as proteases) and having a total of ˜902212 different sequences of aspartic, cysteine, glutamic, metallo, asparagine, serine, threonine and general peptidases catalytics types which are further categorized and include those listed for the following pathways: 2-Oxocarboxylic acid metabolism, ABC transporters, African trypanosomiasis, Alanine, aspartate and glutamate metabolism, Allograft rejection, Alzheimer's disease, Amino sugar and nucleotide sugar metabolism, Amoebiasis, AMPK signaling pathway, Amyotrophic lateral sclerosis (ALS), Antigen processing and presentation, Apoptosis, Arachidonic acid metabolism, Arginine and proline metabolism, Arrhythmogenic right ventricular cardiomyopathy (ARVC), Asthma, Autoimmune thyroid disease, B cell receptor signaling pathway, Bacterial secretion system, Basal transcription factors, beta-Alanine metabolism, Bile secretion, Biosynthesis of amino acids, Biosynthesis of secondary metabolites, Biosynthesis of unsaturated fatty acids, Biotin metabolism, Bisphenol degradation, Bladder cancer, cAMP signaling pathway, Carbon metabolism, Cardiac muscle contraction, Cell adhesion molecules (CAMs), Cell cycle, Cell cycle yeast, Chagas disease (American trypanosomiasis), Chemical carcinogenesis, Cholinergic synapse, Colorectal cancer, Complement and coagulation cascades, Cyanoamino acid metabolism, Cysteine and methionine metabolism, Cytokine-cytokine receptor interaction, Cytosolic DNA-sensing pathway, Degradation of aromatic compounds, Dilated cardiomyopathy, Dioxin degradation, DNA replication, Dorso-ventral axis formation, Drug metabolism—other enzymes, Endocrine and other factor-regulated calcium reabsorption, Endocytosis, Epithelial cell signaling in Helicobacter pylori infection, Epstein-Barr virus infection, Estrogen signaling pathway, Fanconi anemia pathway, Fatty acid elongation, Focal adhesion, Folate biosynthesis, FoxO signaling pathway, Glutathione metabolism, Glycerolipid metabolism, Glycerophospholipid metabolism, Glycosylphosphatidyl-inositol(GPI)-anchor biosynthesis, Glyoxylate and dicarboxylate metabolism, GnRH signaling pathway, Graft-versus-host disease, Hedgehog signaling pathway, Hematopoietic cell lineage, Hepatitis B, Herpes simplex infection, HIF-1 signaling pathway, Hippo signaling pathway, Histidine metabolism, Homologous recombination, HTLV-I infection, Huntington's disease, Hypertrophic cardiomyopathy (HCM), Influenza A, Insulin signaling pathway, Legionellosis, Leishmaniasis, Leukocyte transendothelial migration, Lysine biosynthesis, Lysosome, Malaria, MAPK signaling pathway, Meiosis—yeast, Melanoma, Metabolic pathways, Metabolism of xenobiotics by cytochrome P450, Microbial metabolism in diverse environments, MicroRNAs in cancer, Mineral absorption, Mismatch repair, Natural killer cell mediated cytotoxicity, Neuroactive ligand-receptor interaction, NF-kappa B signaling pathway, Nitrogen metabolism, NOD-like receptor signaling pathway, Non-alcoholic fatty liver disease (NAFLD), Notch signaling pathway, Olfactory transduction, Oocyte meiosis, Osteoclast differentiation, Other glycan degradation, Ovarian steroidogenesis, Oxidative phosphorylation, p53 signaling pathway, Pancreatic secretion, Pantothenate and CoA biosynthesis, Parkinson's disease, Pathways in cancer, Penicillin and cephalosporin biosynthesis, Peptidoglycan biosynthesis, Peroxisome, Pertussis, Phagosome, Phenylalanine metabolism, Phenylalanine, tyrosine and tryptophan biosynthesis, Phenylpropanoid biosynthesis, PI3K-Akt signaling pathway, Plant-pathogen interaction, Platelet activation, PPAR signaling pathway, Prion diseases, Proteasome, Protein digestion and absorption, Protein export, Protein processing in endoplasmic reticulum, Proteoglycans in cancer, Purine metabolism, Pyrimidine metabolism, Pyruvate metabolism, Rap 1 signaling pathway, Ras signaling pathway, Regulation of actin cytoskeleton, Regulation of autophagy, Renal cell carcinoma, Renin-angiotensin system, Retrograde endocannabinoid signaling, Rheumatoid arthritis, RIG-I-like receptor signalling pathway, RNA degradation, RNA transport, Salivary secretion, Salmonella infection, Serotonergic synapse, Small cell lung cancer, Spliceosome, Staphylococcus aureus infection, Systemic lupus erythematosus, T cell receptor signaling pathway, Taurine and hypotaurine metabolism, Terpenoid backbone biosynthesis, TGF-beta signaling pathway, TNF signaling pathway, Toll-like receptor signaling pathway, Toxoplasmosis, Transcriptional misregulation in cancer, Tryptophan metabolism, Tuberculosis, Two-component system, Type I diabetes mellitus, Ubiquinone and other terpenoid-quinone biosynthesis, Ubiquitin mediated proteolysis, Vancomycin resistance, Viral carcinogenesis, Viral myocarditis, Vitamin digestion and absorption Wnt signaling pathway.

Rare molecule fragments can be used to measure peptidase inhibitor of interest included in the MEROPS on-line database for peptidase inhibitors and include a total 133535 different sequences of where a family is a set of homologous peptidase inhibitors with a homology. The homology is shown by a significant similarity in amino acid sequences either to the type inhibitor of the family, or to another protein that has already been shown to be homologous to the type inhibitor, and thus a member of the family. The reference organism for the family are from the group of ovomucoid inhibitor unit 3 (Meleagris gallopavo) aprotinin (Bos taurus), soybean Kunitz trypsin inhibitor (Glycine max), proteinase inhibitor B (Sagittaria sagittifolia), alpha-1-peptidase inhibitor (Homo sapiens), ascidian trypsin inhibitor (Halocynthia roretzi), ragi seed trypsin/alpha-amylase inhibitor (Eleusine coracana), trypsin inhibitor MCTI-1 (Momordica charantia), Bombyx subtilisin inhibitor (Bombyx mori), peptidase B inhibitor (Saccharomyces cerevisiae), marinostatin (Alteromonas sp.), ecotin (Escherichia coli), Bowman-Birk inhibitor unit 1 (Glycine max), eglin c (Hirudo medicinalis), hirudin (Hirudo medicinalis), antistasin inhibitor unit 1 (Haementeria officinalis), streptomyces subtilisin inhibitor (Streptomyces albogriseolus), secretory leukocyte peptidase inhibitor domain 2 (Homo sapiens), mustard trypsin inhibitor-2 (Sinapis alba), peptidase inhibitor LMPI inhibitor unit 1 (Locusta migratoria), potato peptidase inhibitor II inhibitor unit 1 (Solanum tuberosum), secretogranin V (Homo sapiens), BsuPI peptidase inhibitor (Bacillus subtilis), pinA Lon peptidase inhibitor (Enterobacteria phage T4), cystatin A (Homo sapiens), ovocystatin (Gallus gallus), metallopeptidase inhibitor (Bothrops jararaca), calpastatin inhibitor unit 1 (Homo sapiens), cytotoxic T-lymphocyte antigen-2 alpha (Mus musculus), equistatin inhibitor unit 1 (Actinia equina), survivin (Homo sapiens), aspin (Ascaris suum), saccharopepsin inhibitor (Saccharomyces cerevisiae), timp-1 (Homo sapiens), Streptomyces metallopeptidase inhibitor (Streptomyces nigrescens), potato metallocarboxypeptidase inhibitor (Solanum tuberosum), metallopeptidase inhibitor (Dickeya chrysanthemi), alpha-2-macroglobulin (Homo sapiens), chagasin (Leishmania major), oprin (Didelphis marsupialis), metallocarboxypeptidase A inhibitor (Ascaris suum), leech metallocarboxypeptidase inhibitor (Hirudo medicinalis), latexin (Homo sapiens), clitocypin (Lepista nebularis), proSAAS (Homo sapiens), baculovirus P35 caspase inhibitor (Spodoptera litura nucleopolyhedrovirus), p35 homologue (Amsacta moorei entomopoxvirus), serine carboxypeptidase Y inhibitor (Saccharomyces cerevisiae), tick anticoagulant peptide (Ornithodoros moubata), madanin 1 (Haemaphysalis longicornis), squash aspartic peptidase inhibitor (Cucumis sativus), staphostatin B (Staphylococcus aureus), staphostatin A (Staphylococcus aureus), triabin (Triatoma pallidipennis), pro-eosinophil major basic protein (Homo sapiens), thrombostasin (Haematobia irritans), Lentinus peptidase inhibitor (Lentinula edodes), bromein (Ananas comosus), tick carboxypeptidase inhibitor (Rhipicephalus bursa), streptopain inhibitor (Streptococcus pyogenes), falstatin (Plasmodium falciparum), chimadanin (Haemaphysalis longicornis), (Veronica) trypsin inhibitor (Veronica hederifolia), variegin (Amblyomma variegatum), bacteriophage lambda CIII protein (bacteriophage lambda), thrombin inhibitor (Glossina morsitans), anophelin (Anopheles albimanus), Aspergillus elastase inhibitor (Aspergillus fumigatus), AVR2 protein (Passalora fulva), IseA protein (Bacillus subtilis), toxostatin-1 (Toxoplasma gondii), AmFPI-1 (Antheraea mylitta), cvSI-2 (Crassostrea virginica), macrocypin 1 (Macrolepiota procera), HflC (Escherichia coli), oryctin (Oryctes rhinoceros), trypsin inhibitor (Mirabilis jalapa), F1L protein (Vaccinia virus), NvCI carboxypeptidase inhibitor (Nerita versicolor), Sizzled protein (Xenopus laevis), EAPH2 protein (Staphylococcus aureus), and Bowman-Birk-like trypsin inhibitor (Odorrana versabilis). Rare molecule fragments can be used to measure synthetic inhibition of peptidase inhibitor. The afore mentioned data base also includes examples thousands of different small molecule inhibitors that can mimic the inhibitory properties for any member or the above listed family.

Rare molecules of metabolic interest include but are not limited to those that impact the concentration of ACC Acetyl Coenzyme A Carboxylase, Adpn Adiponectin, AdipoR Adiponectin Receptor, AG Anhydroglucitol, AGE Advance glycation end products, Akt Protein kinase B, AMBK pre-alpha-1-microglobulin/bikunin, AMPK 5′-AMP activated protein kinase, ASP Acylation stimulating protein, Bik Bikunin, BNP B-type natriuretic peptide, CCL Chemokine (C-C motif) ligand, CINC Cytokine-induced neutrophil chemoattractant, CTF C-Terminal Fragment of Adiponectin Receptor, CRP C-reactive protein, DGAT Acyl CoA diacylglycerol transferase, DPP-IV Dipeptidyl peptidase-IV, EGF Epidermal growth factor, eNOS Endothelial NOS, EPO Erythropoietin, ET Endothelin, Erk Extracellular signal-regulated kinase, FABP Fatty acid-binding protein, FGF Fibroblast growth factor, FFA Free fatty acids, FXR Farnesoid X receptor a, GDF Growth differentiation factor, GH Growth hormone, GIP Glucose-dependent insulinotropic polypeptide, GLP Glucagon-like peptide-1, GSH Glutathione, GHSR Growth hormone secretagogue receptor, GULT Glucose transporters, GCD59 glycated CD59 (aka glyCD59), HbA1c Hemogloblin A1c, HDL High-density lipoprotein, HGF Hepatocyte growth factor, HIF Hypoxia-inducible factor, HMG 3-Hydroxy-3-methylglutaryl CoA reductase, I-α-I Inter-α-inhibitor, Ig-CTF Immunoglobulin attached C-Terminal Fragment of AdipoR, insulin, IDE Insulin-degrading enzyme, IGF Insulin-like growth factor, IGFBP IGF binding proteins, IL Interleukin cytokines, ICAM Intercellular adhesion molecule, JAK STAT Janus kinase/signal transducer and activator of transcription, JNK c-Jun N-terminal kinases, KIM Kidney injury molecule, LCN-2 Lipocalin, LDL Low-density lipoprotein, L-FABP Liver type fatty acid binding protein, LPS Lipopolysaccharide, Lp-PLA2 Lipoprotein-associated phospholipase A2, LXR Liver X receptors, LYVE Endothelial hyaluronan receptor, MAPK Mitogen-activated protein kinase, MCP Monocyte chemotactic protein, MDA Malondialdehyde, MIC Macrophage inhibitory cytokine, MIP Macrophage infammatory protein, MMP Matrix metalloproteinase, MPO Myeloperoxidase, mTOR Mammalian of rapamycin, NADH Nicotin-amide adenine dinucleotide, NGF Nerve growth factor, NFKB Nuclear factor kappa-light-chain-enhancer of activated B cells, NGAL Neutrophil gelatinase lipocalin, NOS Nitric oxide synthase NOX NADPH oxidase NPY Neuropeptide Yglucose, insulin, proinsulin, c peptide OHdG Hydroxydeoxyguanosine, oxLDL Oxidized low density lipoprotein, P-α-I pre-interleukin-α-inhibitor, PAI-1 Plasminogen activator inhibitor, PAR Protease-activated receptors, PDF Placental growth factor, PDGF Platelet-derived growth factor, PKA Protein kinase A, PKC Protein kinase C, PI3K Phosphatidylinositol 3-kinase, PLA2 Phosphatidylinositol 3-kinase, PLC Phospholipase C, PPAR Peroxisome proliferator-activated receptor, PPG Postprandial glucose, PS Phosphatidylserine, PR Protienase, PYY Neuropeptide like peptide Y, RAGE Receptors for AGE, ROS Reactive oxygen species, S100 Calgranulin, sCr Serum creatinine, SGLT2 Sodium-glucose transporter 2, SFRP4 secreted frizzled-related protein 4 precursor, SREBP Sterol regulatory element binding proteins, SMAD Sterile alpha motif domain-containing protein, SOD Superoxide dismutase, sTNFR Soluble TNF α receptor, TACE TNFα alpha cleavage protease, TFPI Tissue factor pathway inhibitor, TG Triglycerides, TGF β Transforming growth factorβ, TIMP Tissue inhibitor of metalloproteinases, TNF α Tumor necrosis factorsα, TNFR TNF α receptor, THP Tamm-Horsfall protein, TLR Toll-like receptors, TnI Troponin I, tPA Tissue plasminogen activator, TSP Thrombospondin, Uri Uristatin, uTi Urinary trypsin inhibitor, uPA Urokinase-type plasminogen activator, uPAR uPA receptor, VCAM Vascular cell adhesion molecule, VEGF Vascular endothelial growth factor, and YKL-40 Chitinase-3-like protein.

Rare molecules of interest that are highly expressed by pancreas include but are not limited to INS insulin, GLU gluogen, NKX6-1 transcription factor, PNLIPRP1 pancreatic lipase-related protein 1, SYCN syncollin, PRSS1 protease, serine, 1 (trypsin 1) Intracellular, CTRB2 chymotrypsinogen B2 Intracellular, CELA2A chymotrypsin-like elastase family, member 2A, CTRB1 chymotrypsinogen B1 Intracellular, CELA3A chymotrypsin-like elastase family, member 3A Intracellular, CELA3B chymotrypsin-like elastase family, member 3B Intracellular, CTRC chymotrypsin C (caldecrin), CPA1 carboxypeptidase A1 (pancreatic) Intracellular, PNLIP pancreatic lipase, and CPB1 carboxypeptidase B1 (tissue), AMY2A amylase, alpha 2A (pancreatic), and CTFR cystic fibrosis transmembrane conductance regulator.

Rare molecule fragments include those of insulin generated by the following peptidases known to naturally act on insulin; archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, matrix metallopeptidase-9 and others. These fragments include but are not limited to the following sequences: SEQ ID NO: 1 MALWMRLLPLLALLALWGP, SEQ ID NO: 2 MA-LWMRLLPL, SEQ ID NO: 3 ALLALWGPD, SEQ ID NO: 4 AAAFVNQHLCGSHLVEALY-LVCGERGF-FYTPKTR, SEQ ID NO: 5 PAAAFVNQHLCGSHLVEALYLVC, SEQ ID NO: 6 PAAAF-VNQHLCGS, SEQ ID NO: 7 CGSHLVEALYLV, SEQ ID NO: 8 VEAL-YLVC, SEQ ID NO: 9 LVCGERGF, SEQ ID NO: 10 FFYTPK, SEQ ID NO: 11 REAEDL-QVGQVELGGGPGA-GSLQPLALEGSL, SEQ ID NO: 12 REAEDLQVGQVE, SEQ ID NO: 13 LGGGPGAG, SEQ ID NO: 14 SLQPLALEGSL, SEQ ID NO: 15 GIVEQCCTSICSLYQ-LENYCN, SEQ ID NO: 16 GIVEQCCTSICSLY, SEQ ID NO: 17 QLENYCN, and SEQ ID NO: 18 CSLYQLE variation within 75% exact homology. Variations include natural and modified aminoacids.

The rare molecule fragments of insulin of can be used to measure the peptidases acting on insulin based on formation of fragments. This includes the list of natural known peptidase and others added to the biological system. Additional rare molecule fragments of insulin can be used to measure inhibitor for peptidases acting on insulin peptidases based on the formation of fragments. These inhibitor include the c-Terminal fragment of the Adiponectin Receptor, Bikunin, Uristatin and other known natural and synthetic inhibitors of archaelysin, duodenase, calpain-1, ammodytase subfamily M12B peptidases, ALE1 peptidase, CDF peptidase, cathepsin E, meprin alpha subunit, jerdohagin (Trimeresurus jerdonii), carboxypeptidase E, dibasic processing endopeptidase, yapsin-1, yapsin A, PCSK1 peptidase, aminopeptidase B, PCSK1 peptidase, PCSK2 peptidase, insulysin, and matrix metallopeptidase-9 listed in the inhibitor databases.

Rare molecule fragments examples of bioactive proteins and peptides which can be used to measure the presence or absence thereof as an indication of therapeutic effectiveness, stability, usage, metabolism, action on biological pathways (such as actions with proteases, peptidase, enzymes, receptors or other biomolecules), action of inhibition of pathways and other interactions with biological systems. Examples include but are not limited to those listed in databases of approved therapeutic peptides and proteins, such as http://crdd.osdd.net/ as well as other databases of peptides and proteins for dietary supplements, probiotics, food safety, veterinary products, and cosmetics usage. The list of the 467 approved peptide and protein therapies include examples of bioactive proteins and peptides for use in cancer, metabolic disorders, hematological disorders, immunological disorders, genetic disorders, hormonal disorders, bone disorders, cardiac disorders, infectious disease, respiratory disorders, neurological disorders, adjunct therapy, eye disorders, and malabsorption disorder. Bioactive proteins and peptides include those used as anti-thrombins, fibrinolytic, enzymes, antineoplastic agents, hormones, fertility agents, immunosupressive agents, bone related agents, antidiabetic agents, and antibodies

Some specific examples of therapeutic proteins and peptides include glucagon, ghrelin, leptin, growth hormone, prolactin, human placental, lactogen, luteinizing hormone, follicle stimulating hormone, chorionic gonadotropin, thyroid stimulating hormone, adrenocorticotropic hormone, vasopressin, oxytocin, angiotensin, parathyroid hormone, gastrin, buserelin, antihemophilic factor, pancrelipase, insulin, insulin aspart, porcine insulin, insulin lispro, insulin isophane, insulin glulisine, insulin detemir, insulin glargine, immunglobulins, interferon, leuprolide, denileukin, asparaginase, thyrotropin, alpha-1-proteinase inhibitor, exenatide, albumin, coagulation factors, alglucosidase alfa, salmon calcitonin, vasopressin, epidermal growth factor (EGF), cholecystokinin (CCK-8), vaccines, human growth hormone and others. Some new examples of therapeutic proteins and peptides include GLP-1-GCG, GLP-1-GIP, GLP-1, GLP-1-GLP-2, and GLP-1-CCKB

Rare molecules of interest that are highly expressed by adipose tissue include but are not limited to ADIPOQ Adiponectin, C1Q and collagen domain containing, TUSC5 Tumor suppressor candidate 5, LEP Leptin, CIDEA Cell death-inducing DFFA-like effector a, CIDEC Cell death-inducing DFFA-like effector C, FABP4 Fatty acid binding protein 4, adipocyte, LIPE, GYG2, PLIN1 Perilipin 1, PLIN4 Perilipin 4, CSN1S1, PNPLA2, RP11-407P15.2 Protein LOC100509620, L GALS12 Lectin, galactoside-binding, soluble 12, GPAM Glycerol-3-phosphate acyltransferase, mitochondrial, PR325317.1 predicted protein, ACACB Acetyl-CoA carboxylase beta, ACVR1C Activin A receptor, type IC, AQP7 Aquaporin 7, CFD Complement factor D (adipsin)m CSN1S1Casein alpha s1, FASN Fatty acid synthase GYG2 Glycogenin 2 KIF25Kinesin family member 25 LIPELipase, hormone-sensitive PNPLA2 Patatin-like phospholipase domain containing 2 SLC29A4 Solute label family 29 (equilibrative nucleoside transporter), member 4 SLC7A10 Solute label family 7 (neutral amino acid transporter light chain, asc system), member 10, SPX Spexin hormone and TIMP4 TIMP metallopeptidase inhibitor 4.

Rare molecules of interest that are highly expressed by adrenal gland and thyroid include but are not limited to CYP11B2 Cytochrome P450, family 11, subfamily B, polypeptide 2, CYP11B1 Cytochrome P450, family 11, subfamily B, polypeptide 1, CYP17A1 Cytochrome P450, family 17, subfamily A, polypeptide 1, MC2R Melanocortin 2 receptor (adrenocorticotropic hormone), CYP21A2 Cytochrome P450, family 21, subfamily A, polypeptide 2, HSD3B2 Hydroxy-delta-5-steroid dehydrogenase, 3 beta- and steroid delta-isomerase 2, TH Tyrosine hydroxylase, AS3MT Arsenite methyltransferase, CYP11A1 Cytochrome P450, family 11, subfamily A, polypeptide 1, DBH Dopamine beta-hydroxylase (dopamine beta-monooxygenase), HSD3B2 Hydroxy-delta-5-steroid dehydrogenase, 3 beta- and steroid delta-isomerase 2, AKR1B1 Aldo-keto reductase family 1, member B1 (aldose reductase), NOV Nephroblastoma overexpressed, FDX1 Ferredoxin 1, DGKK Diacylglycerol kinase, kappa, MGARP Mitochondria-localized glutamic acid-rich protein, VWA5B2 Von Willebrand factor A domain containing 5B2, C18orf42 Chromosome 18 open reading frame 42, KIAA1024, MAP3K15 Mitogen-activated protein kinase kinase kinase 15, STAR Steroidogenic acute regulatory protein Potassium channel, subfamily K, member 2, NOV nephroblastoma overexpressed, PNMT phenylethanolamine N-methyltransferase, CHGB chromogranin B (secretogranin 1), and PHOX2A paired-like homeobox 2a.

Rare molecules of interest that are highly expressed by bone marrow include but are not limited to DEFA4 defensin alpha 4 corticostatin, PRTN3 proteinase 3, AZU1 azurocidin 1, DEFA1 defensin alpha 1, ELANE elastase, neutrophil expressed, DEFA1B defensin alpha 1B, DEFA3 defensin alpha 3 neutrophil-specific, MS4A3 membrane-spanning 4-domains, subfamily A, member 3 (hematopoietic cell-specific), RNASE3 ribonuclease RNase A family 3, MPO myeloperoxidase, HBD hemoglobin, delta, and PRSS57 protease, serine 57.

Rare molecules of interest that are highly expressed by the brain include but are not limited to GFAP glial fibrillary acidic protein, OPALIN oligodendrocytic myelin paranodal and inner loop protein, OLIG2 oligodendrocyte lineage transcription factor 2, GRIN1glutamate receptor ionotropic, N-methyl D-aspartate 1, OMG oligodendrocyte myelin glycoprotein, SLC17A7 solute label family 17 (vesicular glutamate transporter), member 7, C1orf61 chromosome 1 open reading frame 61, CREG2 cellular repressor of E1A-stimulated genes 2, NEUROD6 neuronal differentiation 6, ZDHHC22 zinc finger DHHC-type containing 22, VSTM2B V-set and transmembrane domain containing 2B, and PMP2 peripheral myelin protein 2.

Rare molecules of interest that are highly expressed by the endometrium, ovary, or placenta include but are not limited to MMP26 matrix metallopeptidase 26, MMP10 matrix metallopeptidase 10 (stromelysin 2), RP4-559A3.7 uncharacterized protein and TRH thyrotropin-releasing hormone.

Rare molecules of interest that are highly expressed by gastrointestinal tract, salivary gland, esophagus, stomach, duodenum, small intestine, or colon include but are not limited to GKN1 Gastrokine 1, GIF Gastric intrinsic factor (vitamin B synthesis), PGA5 Pepsinogen 5 group I (pepsinogen A), PGA3 Pepsinogen 3, group I (pepsinogen A, PGA4 Pepsinogen 4 group I (pepsinogen A), LCT Lactase, DEFA5 Defensin, alpha 5 Paneth cell-specific, CCL25 Chemokine (C-C motif) ligand 25, DEFA6 Defensin alpha 6 Paneth cell-specific, GAST Gastrin, MS4A10 Membrane-spanning 4-domains subfamily A member 10, ATP4A and ATPase, H+/K+ exchanging alpha polypeptide

Rare molecules of interest that are highly expressed by heart or skeletal muscle include but are not limited to NPPB natriuretic peptide B, TNNI3 troponin I type 3 (cardiac), NPPA natriuretic peptide A, MYL7 myosin light chain 7 regulatory, MYBPC3 myosin binding protein C (cardiac), TNNT2 troponin T type 2 (cardiac) LRRC10 leucine rich repeat containing 10, ANKRD1 ankyrin repeat domain 1 (cardiac muscle), RD3L retinal degeneration 3-like, BMP10 bone morphogenetic protein 10, CHRNE cholinergic receptor nicotinic epsilon (muscle), and SBK2 SH3 domain binding kinase family member 2.

Rare molecules of interest that are highly expressed by kidney include but are not limited to UMOD uromodulin, TMEM174 transmembrane protein 174, SLC22A8 solute label family 22 (organic anion transporter) member 8, SLC12A1 solute label family 12 (sodium/potassium/-chloride transporter) member 1, SLC34A1 solute label family 34 (type II sodium/phosphate transporter) member 1, SLC22A12 solute label family 22 (organic anion/urate transporter) member 12, SLC22A2 solute label family 22 (organic cation transporter) member 2, MCCD1 mitochondrial coiled-coil domain 1, AQP2 aquaporin 2 (collecting duct), SLC7A13 solute label family 7 (anionic amino acid transporter) member 13, KCNJ1 potassium inwardly-rectifying channel, subfamily J member 1 and SLC22A6 solute label family 22 (organic anion transporter) member 6.

Rare molecules of interest that are highly expressed by lung include but are not limited to SFTPC surfactant protein C, SFTPA1 surfactant protein A1, SFTPB surfactant protein B, SFTPA2 surfactant protein A2, AGER advanced glycosylation end product-specific receptor, SCGB3A2 secretoglobin family 3A member 2, SFTPD surfactant protein D, ROS1 proto-oncogene 1 receptor tyrosine kinase, MS4A15 membrane-spanning 4-domains subfamily A member 15, RTKN2 rhotekin 2, NAPSA napsin A aspartic peptidase, and LRRN4 leucine rich repeat neuronal 4.

Rare molecules of interest that are highly expressed by liver or gallbladder include but are not limited to APOA2 apolipoprotein A-II, A1BG alpha-1-B glycoprotein, AHSG alpha-2-HS-glycoprotein, F2coagulation factor II (thrombin), CFHR2 complement factor H-related 2, HPX hemopexin, F9 coagulation factor IX, CFHR2 complement factor H-related 2, SPP2 secreted phosphoprotein 2 (24 kDa), C9 complement component 9, MBL2 mannose-binding lectin (protein C) 2 soluble and CYP2A6 cytochrome P450 family 2 subfamily A polypeptide 6.

Rare molecules of interest that are highly expressed by testis or prostate include but are not limited to PRM2 protamine 2 PRM1 protamine 1 TNP1 transition protein 1 (during histone to protamine replacement) TUBA3C tubulin, alpha 3c LELP1late cornified envelope-like proline-rich 1 BOD1L2, biorientation of chromosomes in cell division 1-like 2 ANKRD7 ankyrin repeat domain 7, PGK2 phosphoglycerate kinase 2 AKAP4, A kinase (PRKA) anchor protein 4 TPD52L3, tumor protein D52-like 3, UBQLN3 ubiquilin 3, and ACTL7A actin-like 7A.

### Examples of Rare Cells and Rare Cell Markers

Rare cells are those cells that are present in a sample in relatively small quantities when compared to the amount of non-rare cells in a sample. In some examples, the rare cells are present in an amount of about 10⁻⁸% to about 10⁻²% by weight of a total cell population in a sample suspected of containing the rare cells. The phrase “cell rare molecules” refers to rare molecules that are bound in a cell and may or may not freely circulate in a sample. Such cellular rare molecule include biomolecules useful in medical diagnosis of diseases as above and also include all rare molecules and uses previously described in for cell free rare molecules and those for biomolecules used for measurement of rare cells. The rare cells (cell markers) may be, but are not limited to, malignant cells such as malignant neoplasms or cancer cells; circulating cells, endothelial cells (CD146); epithelial cells (CD326/EpCAM); mesenchymal cells (VIM), bacterial cells, virus, skin cells, sex cells, fetal cells, immune cells (leukocytes such as basophil, granulocytes (CD66b) and eosinophil, lymphocytes such as B cells (CD19,CD20), T cells (CD3, CD4 CD8), plasma cells, and NK cells (CD56), macrophages/monocytes (CD14, CD33), dendritic cells (CD11c, CD123), Treg cells and others), stem cells/precursor (CD34), other blood cells such as progenitor, blast, erythrocytes, thrombocytes, platelets (CD41, CD61, CD62) and immature cells, other cells from tissues such as liver, brain, pancreas, muscle, fat, lung, prostate, kidney, urinary tract, adipose, bone marrow, endometrium, gastrointestinal tract, heart, testis or other for example.

The phrase “population of cells” refers to a group of cells having an antigen or nucleic acid on their surface or inside the cell where the antigen is common to all of the cells of the group and where the antigen is specific for the group of cells. Non-rare cells are those cells that are present in relatively large amounts when compared to the amount of rare cells in a sample. In some examples, the non-rare cells are at least about 10 times, or at least about 10² times, or at least about 10³ times, or at least about 10⁴ times, or at least about 10⁵ times, or at least about 10⁶ times, or at least about 10⁷ times, or at least about 10⁸ times greater than the amount of the rare cells in the total cell population in a sample suspected of containing non-rare cells and rare cells. The non-rare cells may be, but are not limited to, white blood cells, platelets, and red blood cells, for example.

The term “Rare cells markers” include, but are not limited to, cancer cell type biomarkers, cancer bio markers, chemo resistance biomarkers, metastatic potential biomarkers, and cell typing markers, cluster of differentiation (cluster of designation or classification determinant) (often abbreviated as CD) is a protocol used for the identification and investigation of cell surface molecules providing targets for immunophenotyping of cells, for example. Cancer cell type biomarkers include, by way of illustration and not limitation, cytokeratins (CK) (CK1, CK2, CK3, CK4, CK5, CK6, CK7, CK8 and CK9, CK10, CK12, CK 13, CK14, CK16, CK17, CK18, CK19 and CK2), epithelial cell adhesion molecule (EpCAM), N-cadherin, E-cadherin and vimentin, for example. Oncoproteins and oncogenes with likely therapeutic relevance due to mutations include, but are not limited to, WAF, BAX-1, PDGF, JAGGED 1, NOTCH, VEGF, VEGHR, CA1X, MIB1, MDM, PR, ER, SELS, SEM1, PI3K, AKT2, TWIST1, EML-4, DRAFF, C-MET, ABL1, EGFR, GNAS, MLH1, RET, MEK1, AKT1, ERBB2, HER2, HNF1A, MPL, SMAD4, ALK, ERBB4, HRAS, NOTCH1, SMARCB1, APC, FBXW7, IDH1, NPM1, SMO, ATM, FGFR1, JAK2, NRAS, SRC, BRAF, FGFR2, JAK3, RA, STK11, CDH1, FGFR3, KDR, PIK3CA, TP53, CDKN2A, FLT3, KIT, PTEN, VHL, CSF1R, GNA11, KRAS, PTPN11, DDR2, CTNNB1, GNAQ, MET, RB1, AKT1, BRAF, DDR2, MEK1, NRAS, FGFR1, and ROS1, for example.

In certain embodiments, the rare cells may be endothelial cells which are detected using markers, by way of illustration and not limitation, CD136, CD34, CD105/Endoglin, CD145, CD144/VE-cadherin, Tie-2, ESAM, CD145, Cd41 CD136, CD34, CD90, CD31/PECAM-1, VEGFR2/Fik-1, CD202b/TEK, CD56/NCAM, CD73/VAP-2, claudin 5, ZO-1, and vimentin. Metastatic potential biomarkers include, but are limited to, urokinase plasminogen activator (uPA), tissue plasminogen activator (tPA), C terminal fragment of adiponectin receptor (Adiponectin Receptor C Terminal Fragment or Adiponectin CTF), kinases (AKT-PIK3, MAPK), vascular adhesion molecules (e.g., ICAM, VCAM, E-selectin), cytokine signaling (TNF-α, IL-1, IL-6), reactive oxidative species (ROS), protease-activated receptors (PARs), metalloproteinases (TIMP), transforming growth factor (TGF), vascular endothelial growth factor (VEGF), endothelial hyaluronan receptor 1 (LYVE-1), hypoxia-inducible factor (HIF), growth hormone (GH), insulin-like growth factors (IGF), epidermal growth factor (EGF), placental growth factor (PDF), hepatocyte growth factor (HGF), nerve growth factor (NGF), platelet-derived growth factor (PDGF), growth differentiation factors (GDF), VEGF receptor (soluble Flt-1), microRNA (MiR-141), Cadherins (VE, N, E), 5100 Ig-CTF nuclear receptors (e.g., PPARα), plasminogen activator inhibitor (PAI-1), CD95, serine proteases (e.g., plasmin and ADAM, for example); serine protease inhibitors (e.g., Bikunin); matrix metalloproteinases (e.g., MMP9); matrix metalloproteinase inhibitors (e.g., TIMP-1); and oxidative damage of DNA.

Chemoresistance biomarkers include, by way of illustration and not limitation, PL2L piwi like, 5T4, ADLH, β-integrin, α-6-integrin, c-kit, c-met, LIF-R, chemokines (e.g., CXCR7,CCR7, CXCR4), ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24.

The rare molecules from cells may be from any organism, but are not limited to, pathogens such as bacteria, virus, fungus, and protozoa; malignant cells such as malignant neoplasms or cancer cells; circulating endothelial cells; circulating tumor cells; circulating cancer stem cells; circulating cancer mesenchymal cells; circulating epithelial cells; fetal cells; immune cells (B cells, T cells, macrophages, NK cells, monocytes); and stem cells; for example. In some examples of methods in accordance with the principles described herein, the sample to be tested is a blood sample from a mammal such as, but not limited to, a human subject, for example.

Rare cells of interest may be immune cells and include but are not limited to markers for white blood cells (WBC), Tregs (regulatory T cells), B cell, T cells, macrophages, monocytes, antigen presenting cells (APC), dendritic cells, eosinophils, and granulocytes. For example, markers such as, but not limited to CD3, CD4, CD8, CD11c, CD14, CD15, CD16, CD19, CD20, CD31, CD33, CD45, CD52, CD56, CD 61, CD66b, CD123, CTLA-4, immunoglobulin, protein receptors and cytokine receptors and other CD marker that are present on white blood cells can be used to indicate that a cell is not a rare cell of interest.

In particular non-limiting examples of white blood cell markers include CD45 antigen (also known as protein tyrosine phosphatase receptor type C or PTPRC) and originally called leukocyte common antigen is useful in detecting all white blood cells. Additionally, CD45 can be used to differentiate different types of white blood cells that might be considered rare cells. For example, granulocytes are indicated by CD45+, CD15+, or CD16+, or CD66b+; monocytes are indicated by CD45+, CD14+; T lymphocytes are indicated by CD45+, CD3+; T helper cells are indicated by CD45+,CD3+, CD4+; cytotoxic T cells are indicated by CD45+,CD3+, CDS+; B-lymphocytes are indicated by CD45+, CD19+ or CD45+, CD20+; thrombocytes are indicated by CD45+, CD61+; and natural killer cells are indicated by CD16+, CD56+, and CD3-. Furthermore, two commonly used CD molecules, namely, CD4 and CD8, are, in general, used as markers for helper and cytotoxic T cells, respectively. These molecules are defined in combination with CD3+, as some other leukocytes also express these CD molecules (some macrophages express low levels of CD4; dendritic cells express high levels of CD11c, and CD123. These examples are not inclusive of all marker and are for example only.

In some cases, the rare molecule fragment of lymphocytes include proteins and peptides produced as part of lymphocytes such as immunoglobulin chains, major histocompatibility complex (MHC) molecules, T cell receptors, antigenic peptides, cytokines, chemokines and their receptors (e.g, Interluekins, C—X—C chemokine receptors, etc), programmed death-ligand and receptors (Fas, PDL1, and others) and other proteins and peptides that are either parts of the lymphocytes or bind to the lymphocytes.

In other cases the rare cell maybe a stem cell and include but are not limited to the rare molecule fragment of stem markers cells including, PL2L piwi like, 5T4, ADLH, β-integrin, α6 integrin, c-kit, c-met, LIF-R, CXCR4, ESA, CD 20, CD44, CD133, CKS, TRAF2 and ABC transporters, cancer cells that lack CD45 or CD31 but contain CD34 are indicative of a cancer stem cell; and cancer cells that contain CD44 but lack CD24. Stem cell markers include common pluripotency markers like FoxD3, E-Ras, Sall4, Stat3, SUZ12, TCF3, TRA-1-60, CDX2, DDX4, Miwi, Mill GCNF, Oct4, Klf4, Sox2,c-Myc, TIF 1 Piwil, nestin, integrin, notch, AML, GATA, Esrrb, Nr5a2, C/EBPa, Lin28, Nanog, insulin, neuroD, adiponectin, apdiponectin receptor, FABP4, PPAR, and KLF4 and the like.

In other cases the rare cell maybe a pathogen, bacteria, or virus or group thereof which includes, but is not limited to, gram-positive bacteria (e.g., Enterococcus sp. Group B streptococcus, Coagulase-negative staphylococcus sp. Streptococcus viridans, Staphylococcus aureus and saprophyicus, Lactobacillus and resistant strains thereof, for example); yeasts including, but not limited to, Candida albicans, for example; gram-negative bacteria such as, but not limited to, Escherichia coli, Klebsiella pneumoniae, Citrobacter koseri, Citrobacter freundii, Klebsiella oxytoca, Morganella morganii, Pseudomonas aeruginosa, Proteus mirabilis, Serratia marcescens, Diphtheroids (gnb), Rosebura, Eubacterium hallii, Faecalibacterium prauznitzli, Lactobacillus gasseria, Streptococcus mutans, Bacteroides thetaiotaomicron, Prevotella Intermedia, Porphyromonas gingivalis Eubacterium rectale, Lactobacillus amylovorus, Bacillus subtilis, Bifidobacterium longum, Eubacterium rectale, E. eligens, E. dolichum, B. thetaiotaomicron, E. rectale, Actinobacteria, Proteobacteria, B. thetaiotaomicron, Bacteroides Eubacterium dolichum, Vulgatus, B. fragilis, bacterial phyla such as Firmicuties (Clostridia, Bacilli, Mollicutes), Fusobacteria, Actinobacteria, Cyanobacteria, Bacteroidetes, Archaea, Proteobacteria, and resistant strains thereof, for example; viruses such as, but not limited to, HIV, HPV, Flu, and MERSA, for example; and sexually transmitted diseases. In the case of detecting rare cell pathogens, a particle reagent is added that comprises a binding partner, which binds to the rare cell pathogen population. Additionally, for each population of cellular rare molecules on the pathogen, a reagent is added that comprises a binding partner for the cellular rare molecule, which binds to the cellular rare molecules in the population.

As mentioned above, some examples in accordance with the principles described herein are directed to methods of detecting a cell, which include natural and synthetic cells. The cells are usually from a biological sample that is suspected of containing target rare molecules, non-rare cells and rare cells. The samples may be biological samples or non-biological samples. Biological samples may be from a mammalian subject or a non-mammalian subject. Mammalian subjects may be, e.g., humans or other animal species.

### Kits for Conducting Methods

The apparatus and reagents for conducting a method in accordance with the principles described herein may be present in a kit useful for conveniently performing the method. In one embodiment, a kit comprises in packaged combination modified affinity agent one for each different rare molecule acid to be isolated. The kit may also comprise one or more, cell affinity agent for cell containing the rare molecules, the porous matrix, optional capture particles, solution for spraying, filtering and reacting the mass labels, droplet generators, capillaries nozzles for droplet formation, capillary channels for dilution, concentration or routing of solutions, droplets and molecules, solutions for forming droplets, solutions for breaking droplets The composition may contain labeled particles or capture particle entities, for example, as described above. Porous matrix, liquid holding wells, porous matrix and droplet generators can be in housing where the house can have vents, capillaries, chambers, liquid inlets and outlets. A solvent can be applied to droplet generators, wells and porous matrix. The porous matrix can be removable.

Depending on method for analysis of rare molecules of selected, reagents discussed in more detail herein below, may or may not be used to treat the samples during, prior or after the extract molecules from the rare cells and cell free samples.

The relative amounts of the various reagents in the kits can be varied widely to provide for concentrations of the reagents that substantially optimize the reactions that need to occur during the present methods and further to optimize substantially the sensitivity of the methods. Under appropriate circumstances one or more of the reagents in the kit can be provided as a dry powder, usually lyophilized, including excipients, which on dissolution will provide for a reagent solution having the appropriate concentrations for performing a method in accordance with the principles described herein. The kit can further include a written description of a method utilizing reagents in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The spray solvent can be any spray solvent employed in electrospray mass spectroscopy. In some examples, solvents for electrospray ionization include, but are not limited to, polar organic compounds such as, e.g., alcohols (e.g., methanol, ethanol and propanol), acetonitrile, dichloromethane, dichloroethane, tetrahydrofuran, dimethylformamide, dimethyl sulphoxide, and nitromethane; non-polar organic compounds such as, e.g., hexane, toluene, cyclohexane; and water, for example, or combinations of two or more thereof. Optionally, the solvents may contain one or more of an acid or a base as a modifier (such as, volatile salts and buffer, e.g., ammonium acetate, ammonium biocarbonate, volatile acids such as formic acid, acetic acids or trifluoroacetic acid, heptafluorobutyric acid, sodium dodecyl sulphate, ethylenediamine tetraacetic acid, and non-volatile salts or buffers such as, e.g., chlorides and phosphates of sodium and potassium, for example.

In many examples, the sample is contacted with an aqueous phase prior to forming an emulsion. The aqueous phase may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxane, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume. In some examples, the pH for the aqueous medium is usually a moderate pH. In some examples, the pH of the aqueous medium is about 5 to about 8, or about 6 to about 8, or about 7 to about 8, or about 5 to about 7, or about 6 to about 7, or physiological pH. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE.

Cell and/or droplet lysis reagents are those that involve disruption of the integrity of the cellular membrane with a lytic agent, thereby releasing intracellular contents of the cells. Numerous lytic agents are known in the art. Lytic agents that may be employed may be physical and/or chemical agents. Physical lytic agents include, blending, grinding, and sonication, and combinations or two or more thereof, for example. Chemical lytic agents include, but are not limited to, non-ionic detergents, anionic detergents, amphoteric detergents, low ionic strength aqueous solutions (hypotonic solutions), bacterial agents, and antibodies that cause complement dependent lysis, and combinations of two or more thereof, for example, and combinations or two or more of the above. Non-ionic detergents that may be employed as the lytic agent include both synthetic detergents and natural detergents.

The nature and amount or concentration of lytic agent employed depends on the nature of the cells, the nature of the cellular contents, the nature of the analysis to be carried out, and the nature of the lytic agent, for example. The amount of the lytic agent is at least sufficient to cause lysis of cells to release contents of the cells. In some examples, the amount of the lytic agent is (percentages are by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

Removal of lipids, platelets, and non rare cells may be carried out using, by way of illustration and not limitation, detergents, surfactants, solvents, and binding agents, and combinations of two or more of the above, for example, and combinations of two or more thereof. The use of a surfactant or a detergent as a lytic agent as discussed above accomplishes both cell lysis and removal of lipids. The amount of the agent for removing lipids is at least sufficient to remove at least about 50%, or at least about 60%, or at least about 70%, or at least about 80%, or at least about 90%, or at least about 95% of lipids from the cellular membrane. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, it may be desirable to remove or denature proteins from the cells, which may be accomplished using a proteolytic agent such as, but not limited to, proteases, heat, acids, phenols, and guanidinium salts, and combinations of two or more thereof, for example. The amount of the proteolytic agent is at least sufficient to degrade at least about 50%, or at least about 60%, or at least about 70%, or at least about 80%, or at least about 90%, or at least about 95% of proteins in the cells. In some examples the amount of the lytic agent is (percentages by weight) about 0.0001% to about 0.5%, about 0.001% to about 0.4%, about 0.01% to about 0.3%, about 0.01% to about 0.2%, about 0.1% to about 0.3%, about 0.2% to about 0.5%, about 0.1% to about 0.2%, for example.

In some examples, samples are collected from the body of a subject into a suitable container such as, but not limited to, a cup, a bag, a bottle, capillary, or a needle, for example. Blood samples may be collected into VACUTAINER® containers, for example. The container may contain a collection medium into which the sample is delivered. The collection medium is usually a dry medium and may comprise an amount of platelet deactivation agent effective to achieve deactivation of platelets in the blood sample when mixed with the blood sample.

Platelet deactivation agents can be added to the sample such as, but are not limited to, chelating agents such as, for example, chelating agents that comprise a triacetic acid moiety or a salt thereof, a tetraacetic acid moiety or a salt thereof, a pentaacetic acid moiety or a salt thereof, or a hexaacetic acid moiety or a salt thereof. In some examples, the chelating agent is ethylene diamine tetraacetic acid (EDTA) and its salts or ethylene glycol tetraacetate (EGTA) and its salts. The effective amount of platelet deactivation agent is dependent on one or more of the nature of the platelet deactivation agent, the nature of the blood sample, level of platelet activation and ionic strength, for example. In some examples, for EDTA as the anti-platelet agent, the amount of dry EDTA in the container is that which will produce a concentration of about 1.0 to about 2.0 mg/mL of blood, or about 1.5 mg/mL of the blood. The amount of the platelet deactivation agent is that which is sufficient to achieve at least about 90%, or at least about 95%, or at least about 99% of platelet deactivation.

Moderate temperatures are normally employed, which may range from about 5° C. to about 70° C. or from about 15° C. to about 70° C. or from about 20° C. to about 45° C., for example. The time period for an incubation period is about 0.2 seconds to about 6 hours, or about 2 seconds to about 1 hour, or about 1 to about 5 minutes, for example. These temperature can be used to reverse fixations or other reactions.

In many examples, the above combination is provided in an aqueous medium, which may be solely water or which may also contain organic solvents such as, for example, polar aprotic solvents, polar protic solvents such as, e.g., dimethylsulfoxide (DMSO), dimethylformamide (DMF), acetonitrile, an organic acid, or an alcohol, and non-polar solvents miscible with water such as, e.g., dioxene, in an amount of about 0.1% to about 50%, or about 1% to about 50%, or about 5% to about 50%, or about 1% to about 40%, or about 1% to about 30%, or about 1% to about 20%, or about 1% to about 10%, or about 5% to about 40%, or about 5% to about 30%, or about 5% to about 20%, or about 5% to about 10%, by volume. In some examples, the pH for the aqueous medium is usually a moderate pH. In some examples the pH of the aqueous medium is about 5 to about 8, or about 6 to about 8, or about 7 to about 8, or about 5 to about 7, or about 6 to about 7, or physiological pH, for example. Various buffers may be used to achieve the desired pH and maintain the pH during any incubation period. Illustrative buffers include, but are not limited to, borate, phosphate (e.g., phosphate buffered saline), carbonate, TRIS, barbital, PIPES, HEPES, MES, ACES, MOPS, and BICINE, for example.

An amount of aqueous medium employed is dependent on a number of factors such as, but not limited to, the nature and amount of the sample, the nature and amount of the reagents, the stability of rare cells, and the stability of rare molecules, for example. In some examples in accordance with the principles described herein, the amount of aqueous medium per 10 mL of sample is about 5 mL to about 100 mL, or about 5 mL to about 80 mL, or about 5 mL to about 60 mL, or about 5 mL to about 50 mL, or about 5 mL to about 30 mL, or about 5 mL to about 20 mL, or about 5 mL to about 10 mL, or about 10 mL to about 100 mL, or about 10 mL to about 80 mL, or about 10 mL to about 60 mL, or about 10 mL to about 50 mL, or about 10 mL to about 30 mL, or about 10 mL to about 20 mL, or about 20 mL to about 100 mL, or about 20 mL to about 80 mL, or about 20 mL to about 60 mL, or about 20 mL to about 50 mL, or about 20 mL to about 30 mL, for example.

Where one or more of the rare nucleic acids are part of a cell, the aqueous medium may also comprise a lysing agent for lysing of cells. A lysing agent is a compound or mixture of compounds that disrupt the integrity of the matrixes of cells thereby releasing intracellular contents of the cells. Examples of lysing agents include, but are not limited to, non-ionic detergents, anionic detergents, amphoteric detergents, low ionic strength aqueous solutions (hypotonic solutions), bacterial agents, aliphatic aldehydes, and antibodies that cause complement dependent lysis, for example. Various ancillary materials may be present in the dilution medium. All of the materials in the aqueous medium are present in a concentration or amount sufficient to achieve the desired effect or function.

In some examples, it may be desirable to fix the nucleic acids, proteins or cells of the sample. Fixation immobilizes the nucleic acids and preserves the nucleic acids structure and maintains the cells in a condition that closely resembles the cells in an in vivo-like condition and one in which the antigens of interest are able to be recognized by a specific affinity agent. The amount of fixative employed is that which preserves the nucleic acids or cells but does not lead to erroneous results in a subsequent assay. The amount of fixative depends on one or more of the nature of the fixative and the nature of the cells, for example. In some examples, the amount of fixative is about 0.05% to about 0.15% or about 0.05% to about 0.10%, or about 0.10% to about 0.15%, for example, by weight. Agents for carrying out fixation of the cells include, but are not limited to, cross-linking agents such as, for example, an aldehyde reagent (such as, e.g., formaldehyde, glutaraldehyde, and paraformaldehyde); an alcohol (such as, e.g., C₁-C₅ alcohols such as methanol, ethanol and isopropanol); a ketone (such as a C₃-C₅ ketone such as acetone); for example. The designations C₁-C₅ or C₃-C₅ refer to the number of carbon atoms in the alcohol or ketone. One or more washing steps may be carried out on the fixed cells using a buffered aqueous medium.

In examples in which fixation is employed, extraction of nucleic acids can include a procedure for de-fixation prior to amplification. De-fixation may be accomplished employing, by way of illustration and not limitation, heat or chemicals capable of reversing cross-linking bonds, or a combination of both, for example.

In some examples utilizing the techniques, it may be necessary to subject the rare cells to permeabilization. Permeabilization provides access through the cell membrane to nucleic acids of interest. The amount of permeabilization agent employed is that which disrupts the cell membrane and permits access to the nucleic acids. The amount of permeabilization agent depends on one or more of the nature of the permeabilization agent and the nature and amount of the rare cells, for example. In some examples, the amount of permeabilization agent by weight is about 0.1% to about 0.5%, or about 0.1% to about 0.4%, or about 0.1% to about 0.3%, or about 0.1% to about 0.2%, or about 0.2% to about 0.5%, or about 0.2% to about 0.4%, or about 0.2% to about 0.3%, for example. Agents for carrying out permeabilization of the rare cells include, but are not limited to, an alcohol (such as, e.g., C₁-C₅ alcohols such as methanol and ethanol); a ketone (such as a C₃-C₅ ketone such as acetone); a detergent (such as, e.g., saponin, Triton® X-100, and Tween®-20); for example. One or more washing steps may be carried out on the permeabilized cells using a buffered aqueous medium.

The following examples further describe the specific embodiments of the invention by way of illustration and not limitation and are intended to describe and not to limit the scope of the invention. Parts and percentages disclosed herein are by volume unless otherwise indicated.

### EXAMPLES

All chemicals may be purchased from the Sigma-Aldrich Company (St. Louis Mo.) unless otherwise noted. Abbreviations:

min=minute(s)
μm=micron(s)
mL=milliliter(s)
mg=milligrams(s)
μg=microgram(s)
w/w=weight to weight
RT=room temperature
hr=hour(s)
QS=quantity sufficient
Ab=antibody
mAb=monoclonal antibody
vol=volume
MW=molecular weight
wt.=weight
Phosphate buffered saline (PBS)=3.2 mM Na₂HPO₄, 0.5 mM KH₂PO₄, 1.3 mM KCl, and 135 mM NaCl at pH 7.4
PBS-EDTA buffer=0.5M EDTA in PBS
Capture particles=Magnetic beads BioMag® hydroxyl silica micro particles (46.2 mg/mL, 1.5 μm) with streptavidin (Bangs Lab Inc.)
Magnet=Dynal magnetic particle concentrator
Label particles=Silica amine label particle=Propylamine-functionalized silica nano-particles 200 μm, mesoporous pore sized 4 nm
Porous Matrix=WHATMAN® NUCLEOPORE™ Track Etch matrix, 25 mm diameter and 8.0 and 1.0 μM pore sizes

### Example 1

### Sequencing of Genes by Mass Label Release

The most practical way to get enough genetic material whether RNA or DNA for single cell detection is to obtain purified single cells, in this case SKBR human breast cancer cells.

Alternatively, one can isolate particular subtypes of RNA or DNA from these cells released into circulation. For example, SKBR cells can be lyzed and cell free DNA isolated which is typically fragments to 85 to 230 bp. The observed reference range for normal cfDNA in blood is between 200 ng and 40 μg/10 mL healthy persons and patient have 58 and 5317 ng/ml. The disease cfDNA to background cfDNA is therefore 0.01%. Similar a cancer cell in blood can be 1 to 300 cells per blood tube and 0.1% purity after cell filtration. These case of low concentration and purity requires targeted purification of genes of interest by capture on particles and washing on to particles prior to step pre-amplification.

A method of removing the cell or cell free nucleic acids by size exclusion filtration droplet were diluted in PBS, and filtered through as filtration process as previously described in (Using Automated Microfluidic Filtration and Multiplex Immunoassay Magbanua M J M, Pugia M, Lee J S, Jabon M, Wang V, et al. (2015) A Novel Strategy for Detection and Enumeration of Circulating Rare Cell Populations in Metastatic Cancer Patients Using Automated Microfluidic Filtration and Multiplex Immunoassay. PLoS ONE 10(10)). In this example, SBKR cells at 10² cells/blood tube were stained with label particle for demonstration of cellular nucleic acids capture and lysed and bound to particles for cell free nucleic acids capture. The only change to the process was to use a vacuum filtration unit (Biotek Inc) for a standard ELISA plate fitted with the standard.

A porous matrix with 8.0 μm pores was used for the cell isolation and 10.1 μm pores for the gene or 1.0 μm if captured on a particle or 8 μm for a droplet library. The cells in this library were ˜10 μm diameter (5 to 30 μm range), nucleic acids cDNA particle were ˜20 nm diameter (10 to 400 nm range), and protein capture with label particles were ˜1.5 μm diameter (1 to 2 μm range), droplets with protein capture with label particles were ˜10 μm diameter (5 to 20 μm range). Cell clusters were ˜75 μm average diameter (50 to 300 μm range). Each droplet library contained 10⁴ to 10⁶ unique molecules in full droplets and 10⁶ to 10⁹ empty droplets.

Cell, droplets, particles and genes were filtrated into a porous matrix, sample on the porous matrix was subjected to a negative mBar, that is, a decrease greater than about −100 mBar from atmospheric pressure. The vacuum applied varied from −10 to −100 mBar during filtration. The droplets in a diluted sample was placed into the filtration station without mixing and the sample was filtered through the porous matrix. Th cell diameter for ˜20 μm, ˜100 nm diameter for cDNA, were ˜5 um diameter for nucleic acid capture particles and were ≣20 μm diameter for droplets. After the liquid was removed by vacuum filtration, a surfactant, in this case 0.5% Triton X 100 in PBS was added to wash the unbound materials.

Targeted purification of the genes of interest was done by capture oligos on particles such as magnetic particles or surfaces. In this chemistry, oligonucleotides linked to the particles are used to bind the target gene through a complementary oligonucleotides and remaining background materials are washed away. The complementary oligonucleotides have to be heated to hybridize to the target. At this point the genetic product is a clean material and can be archived for later use or amplification.

The isolated material is then amplified for sequencing of specific gene (in this case CK19). In this case the mRNA was converted to cDNA by reverse transcriptase. In some cases, multiple target genes are captured by different oligo particles in separate wells. Each well is washed remove other gene materials. This eliminates the need for bar coding. This material also can be measured by traditional analysis such as polymerase chain reaction (PCR), Droplet digital PCR or next generation sequencing for comparisons.

The material is then reacted for mass label sequencing of the specific gene (in this case CK19). PCR Amplification with MS label-termination was done using a sanger sequencing protocol. Mass spectrometry was able to detect 10⁴ to 10⁶ copies of genes products at high purity of target (>80%) and a small sample volume (1 μL), as the material is amplified to 1 nM concentration to achieve a detectable MS label concentration. This required a 10⁶ copy number amplification, therefore a PCR amplification is done for 20 cycles followed by addition of MS label-terminator Sanger sequencing for primer elongation utilizing chain terminator ddNTPs with a different MS label off unique mass for the four base pairs. In some examples, MS label-terminator sanger sequencing utilizes labelling of the chain terminator ddNTPs, which permits sequencing in a single reaction, and, each of the four deoxynucleotide chain terminators is labelled with a different MS label that has unique mass. The reaction mixture, primer, DNA template with the ddNTPs with the four different Mass labels, DNA polymerase, and dNTPs (dATP, dCTP, dGTP, and dTTP) are used in the reactions.

The invention was demonstrated by detection of mass label which are releasable by breaking a bond and in this case using an acetal bond that releases the mass label at acidic pH after adding internal standard mass label were released and detected in the mass spectrometer. A digital mass spectrometer sequencing read out was demonstrated by identification of elongation chain length by mass and compare to the expected to determine terminal nucleic sequence locations. Acidification of spray solvent and release MS to identify nucleotide at each sequence terminal locations by mass loss. Determine total released mass labels for expression level. Both copy number concentration and targeted mass label release sequencing were demonstrated for short reads of 5 to 20 bp, are (1500 to 6153 da down to 400 da),

Since, one cell nucleated only contains about ˜3 pg of genomic DNA or few copies of sequence. This low concentration requires highly accurate pre-amplification to have enough material for a single cell to further react for mass label sequencing. The amplification must have an extremely low error rate (high fidelity) to prevent the propagation of error in sequence. For DNA, using a method like multiple displacement amplification (MDA) allow isothermal high fidelity pre-amplification due to 3′-5′ proof reading activity which reduces the amplification error rate to 1 in 10⁶-10⁷. Doing this pre-amplification allows 10⁵ more copies or 3.3 μg of genomic DNA material in cDNA form of ˜300 bp. This material is now ready for step 2 for targeted capture. Since, one cell nucleated only contains ˜10-30 pg total RNA and there are 10⁵ copies of RNA per cell, there is enough material to further purify prior to reverse transcriptase (RT) amplification.

The genetic material contains a lot of non-essential code therefore target purification by capture on particles and washing was needed for this method. A genome of a single cell is 3 billon base pairs (from the 23 pairs of chromosome). Typical gene of interest are 300 bp or less. Typical sets of gene panels of interest are 100 genes or less. The total for 100 gene panel can only be 150,000 bp or 0.005% of entire genetic material. This impurity makes the amplification for sequencing, highly inefficient as polymerase must work through all material and have an effective error rate of 4% after 30 cycles. Therefore a targeted purification of genes of interest by capture on particles and washing on to particles is used.

All patents, patent applications and publications cited in this application including all cited references in those patents, applications and publications, are hereby incorporated by reference in their entirety for all purposes to the same extent as if each individual patent, patent application or publication were so individually denoted.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention.

What is claimed is:

1. A method of gene sequencing by mass spectroscopy said method comprising release and detection of mass labeled nucleic acids.

2. The method of claim 1, wherein the mass labels are attached as chain terminators to the nucleic acid.

3. The method of claim 1, wherein the mass labels include a breakable bond.

4. The method of claim 1, wherein the mass labels are optimized for ionization and detection by mass spectrometry.

5. The method of claim 1, wherein said mass labels are attached to the nucleic acids by conventional organic synthesis.

6. The method of claim 5, wherein said synthesis includes amplification of the nucleic acids and the releasable mass label terminators are 2′,3′ dideoxynucleotides (ddNTPs).

7. The method of claim 5, wherein said synthesis of nucleic acids include lengths of <3000 base pairs.

8. The method of claim 1, wherein the mass labels are attached to nucleic acids that are isolated.

9. The method of claim 8, wherein said nucleic acids are isolated by capture and purification.

10. The method of claim 8, wherein said nucleic acids are captured on particles or contained inside droplets.

11. The method of claim 8, wherein said captured nucleic acids are inside cells or released from cells.

12. The method of claim 10, wherein said captured nucleic acids are isolated by size exclusion filtration, or captured on particles.

13. The method of claim 1, wherein said detection further requires release from a liquid holding area for mass spectroscopic analysis.

14. The method of claim 1, wherein said measurement of nucleic acids by mass label can serve as a bar code to identify the presence of unique analyte or as a signal to quantitate the amount of analyte.

15. The method of claim 1, wherein said detection requires determining the number of base pairs in nucleic acids by mass and release of the mass label-terminator to identify the terminal nucleotide in the nucleic acids.

16. The method of claim 1, wherein said method further includes the following steps:
(a) isolation of the nucleic acid;
(b) amplification of the nucleic acid and chain termination with a 2′,3′ dideoxynucleotides as the releasable mass label terminator;
(c) identification of the number of base pairs in the products by mass and;
(d) release and identification of the mass label-terminator to identify the terminal nucleotide in the sequence.
